(19) United States
(12) Patent Application Publication
(10) Pub. No.: US 2024/0127918 A1
(43) Pub. Date: Apr. 18, 2024

(54) HEALTHCARE PLAN ESTABLISHMENT AND IMPLEMENTATION

(71) Applicant: Ping-Chun Wang, New Taipei City (TW)

(72) Inventor: Ping-Chun Wang, New Taipei City (TW)

(21) Appl. No.: 17/964,065

(22) Filed: Oct. 12, 2022

(51) Int. Cl. G16H 20/00; G16H 10/20; G16H 10/60; H04L 67/12

(52) U.S. Cl. CPC G16H 20/00; G16H 10/20; G16H 10/60; H04L 67/12

(57) ABSTRACT

A healthcare method provides services to establish portfolios of a healthcare plan according to user's health condition for managing their requirements for a period of time. These requirements may be classified to a variety of elements such as exercise, nutrients, diet, medical treatment and other services. Generally, most of people are very busy on their business and suffering from a lot of stress, it is easily to make them accumulate negative thinking, mood, health condition and dietary habit. It may make them neglect requirements of healthcare. Moreover, few people are interested in implementations of healthcare knowledge such as exercise, nutrition, disease, medical treatment and others. They usually like to google latest healthcare knowledge and keep it in their mind. However, these implementations are sophisticated and connected with each other. It is hard to be completely and accurately implemented for us. So, it may be an efficient way to carry out these implementations by means of the healthcare method.

### BACKGROUND

The environment we live in is convenient, rich of knowledge and abundant supply of food and drinks, people are easy to find and buy food and drinks without a careful consideration on their choices. Additionally, many manufacturers and restaurants of food and drinks intentionally develop a series of delicious products for their consumers. Most of their products may be refined, processed or overcooked, and focus on fancy appearance, delicacy and tasty favor. It's a tendency that their consumers are pursuing the diversity of delicious food and drinks.

The current society is competitive and high efficiency, it makes people may be very busy on their business, suffering from a lot of stress and lack of information on their dietary criterion. They may neglect arrangements on their dietary plans and only choose fast food, junk food, snack and delicacy for their meals in order to get rid of their stress, exercise as well. Generally, many people are easy to eat over 3 meals a day and get too many calories, especially refining sugar and trans fats. They seldom pay more attention on daily nutrients and exercise. It may gradually influence their health.

Eventually, people can google a lot of information about daily nutrients, suggestions of exercise and dietary notice which many experts share, they may only keep in their mind or arrange a dietary plan by using a form. It may be inaccurate and inefficient way to carry out a purpose making people healthy and preventing from a lot of diseases. The method is developed to accurately and efficiently manage individual dietary and exercise plan based on personal health status. It may prevent from wrongdoing on the executions of the individual dietary plan.

### SUMMARY

The document describes method and system for providing services to build up portfolios of healthcare plan for a user for managing their requirements for a period of time. The system may install a diversity of proprietary modules and databases for processing data collected from user's behavior through a plurality of clients and sensors. These proprietary modules and databases may include technologies of image recognition, medical diagnosis, 3D (three dimensions) simulation, action predictions, deep learning and other applications. First of all, the system may be intended to request a user to make a registration before a usage of healthcare services, especially their health status. It may establish portfolios of healthcare plan for the user according to their health status.

The system may be intended to build up databases of a variety of organizations, hospitals, supermarkets, restaurants and stores for providing services to users. While they are moving to these places, their clients may be configured to transmit their location information to the system for confirming whether they arrive at these places. When the system determines they go into these places based on their location information, it may be intended to make communication sessions with user's client, servers, clients and sensors in these places. The user may be notified by an event of healthcare plan to use services in these places. The system may be intended to monitor user's behavior through clients and sensors in these places and illustrate upcoming events on a display of a user's client.

The method may be intended to request clients and sensors in the place collecting a lot of information from user's behavior in the area where a user locates. When a user decides engaging in an event, it may provide suggestions to a user in the early stage of the event and then confirm their decision about the event with them. The system may start receiving a lot of information from servers in the place while they are engaging in the event. It may be intended to analyze the lot of information in order to make calculations for user's health status and the lot of information. If it detects that a result of the calculations is different from the target of the event or the user does wrongdoings, it may determine interrupting or terminating user's engagement. If a user makes wrongdoings during the event, it may intentionally demonstrate exact processes of the event to them through clients and sensors in the area where a user locates.

The present disclosure describes the following implementations, a healthcare plan establishment and implementation method comprises initiating, by an event of a healthcare plan, a plurality of clients and sensors In an area where a user arrives at. The method comprises detecting, by the plurality of clients and sensors, the user starts to engage in the event of the healthcare plan. The method comprises illustrating user's health status and the scenario of the event in the early stage of the event. The method comprises requesting, by the plurality of clients and sensors, a predetermined information of executing the event in the early stage of the event. The method comprises estimating the user's health status and the predetermined information in the early stage of the event. The method comprises providing a suggestion based on the estimation of the user's health status and the predetermined information in the early stage of the event. The method comprises confirming a decision with the user responsive to the acknowledgement of the suggestion from the user in the early stage of the event. The method comprises demonstrating, by the plurality of clients and sensors, entire process of the event responsive to the confirmation of the user in the early stage of the event. The method comprises configuring the plurality of clients and sensors involving in the event to periodically collect a diversity of data from the user's behavior of engaging in the event. The method comprises analyzing the diversity of data collected from the user's behavior of engaging in the event. The method comprises calculating the diversity of data and variety of values representing the user's health status. The method comprises updating the user's health status after the calculations for the diversity of data and variety of values.

The method further comprises defining a target for each event in the user's healthcare plan based on the information of the user's health status and database of technologies, creating processes and scenarios of events based on the information of the user's health status and database of technologies, and adjusting dynamically an order of processes or scenario of events while the user is engaging in the events.

The method further comprises receiving periodically current location information provided by location-aware components of user's client, while the user's client is executing the event of the healthcare plan, forming a list of venues suitable to carry out the event by determining the user arrives in a predetermined area enclosing the venues, and illustrating a suggestion about an order of executing events in the venues of the list in the predetermined area to the user.

The method further comprises detecting the plurality of clients and sensors unable to collect information from the user's behavior while the user is engaging in the event, interrupting the user's engagement for illustrating which part of the event the plurality of clients and sensors are unable to collect the information from, and requesting the user to reply the information in order to continue the user's engagement in the event.

The method further comprises detecting the user's health status is deviating from the purpose of the event based on the series of information while the user is engaging in the event, notifying the user about the deviation from the purpose of the event in order to interrupt the user's engagement, performing a demonstration or termination of procedures of the event based on the analysis of the deviation, and modifying the healthcare plan if the deviation is caused by improper processes of the event.

The method further comprises Initiating a plurality of instruments, clients and sensors In the area where the user locates, measuring, by the plurality of instruments, clients and sensors, the user's health status after the event is completed, and updating the user's health status and healthcare plan in response to the measurement for the user's health status.

The non-transitory computer-readable medium storing instructions that, when executed by a processor, cause the processor to implement the method comprises initiating, by an event of a healthcare plan, a plurality of clients and sensors In an area where a user arrives at. The method comprises detecting, by the plurality of clients and sensors, the user starts to engage in the event of the healthcare plan. The method comprises illustrating user's health status and the scenario of the event in the early stage of the event. The method comprises requesting, by the plurality of clients and sensors, a predetermined information of executing the event in the early stage of the event. The method comprises estimating the user's health status and the predetermined information in the early stage of the event. The method comprises providing a suggestion based on the estimation of the user's health status and the predetermined information in the early stage of the event. The method comprises confirming a decision with the user responsive to the acknowledgement of the suggestion from the user in the early stage of the event. The method comprises demonstrating, by the plurality of clients and sensors, entire process of the event responsive to the confirmation of the user in the early stage of the event. The method comprises configuring the plurality of clients and sensors involving in the event to periodically collect a diversity of data from the user's behavior of engaging in the event. The method comprises analyzing the diversity of data collected from the user's behavior of engaging in the event. The method comprises calculating the diversity of data and variety of values representing the user's health status. The method comprises updating the user's health status after the calculations for the diversity of data and variety of values.

The system comprises one or more processors, and one or more non-transitory computer-readable media storing instructions that, when executed by the one or more processors, cause the system to perform operations comprises initiating, by an event of a healthcare plan, a plurality of clients and sensors In an area where a user arrives at. The method comprises detecting, by the plurality of clients and sensors, the user starts to engage in the event of the healthcare plan. The method comprises illustrating user's health status and the scenario of the event in the early stage of the event. The method comprises requesting, by the plurality of clients and sensors, a predetermined information of executing the event in the early stage of the event. The method comprises estimating the user's health status and the predetermined information in the early stage of the event. The method comprises providing a suggestion based on the estimation of the user's health status and the predetermined information in the early stage of the event. The method comprises confirming a decision with the user responsive to the acknowledgement of the suggestion from the user in the early stage of the event. The method comprises demonstrating, by the plurality of clients and sensors, entire process of the event responsive to the confirmation of the user in the early stage of the event. The method comprises configuring the plurality of clients and sensors involving in the event to periodically collect a diversity of data from the user's behavior of engaging in the event. The method comprises analyzing the diversity of data collected from the user's behavior of engaging in the event. The method comprises calculating the diversity of data and variety of values representing the user's health status. The method comprises updating the user's health status after the calculations for the diversity of data and variety of values.

### DESCRIPTION OF THE DRAWINGS

FIG. 1 is a viewpoint of cameras and sensors capturing images and information from people.

FIG. 2 is a block diagram of a system that may be used to implement the methods and systems described in the document.

FIG. 3 is a block diagram illustrating the healthcare module of the server.

FIG. 4 is a block diagram illustrating the mathematics module of the server.

FIG. 5 is a block diagram illustrating the control module of the server.

FIG. 6 is a block diagram illustrating the membership module of the server.

FIG. 7 is a flowchart of a process for implementing healthcare method.

FIG. 8 is a block diagram illustrating components of a computing device that may be used to implement the methods and systems described in the document, as a server or plurality of servers.

FIG. 9 is a block diagram illustrating components of a computing device that may be used to implement the methods and systems described in the document, as a client or plurality of clients.

Like reference symbols in the variety drawings indicate like elements.

### DETAILED DESCRIPTION

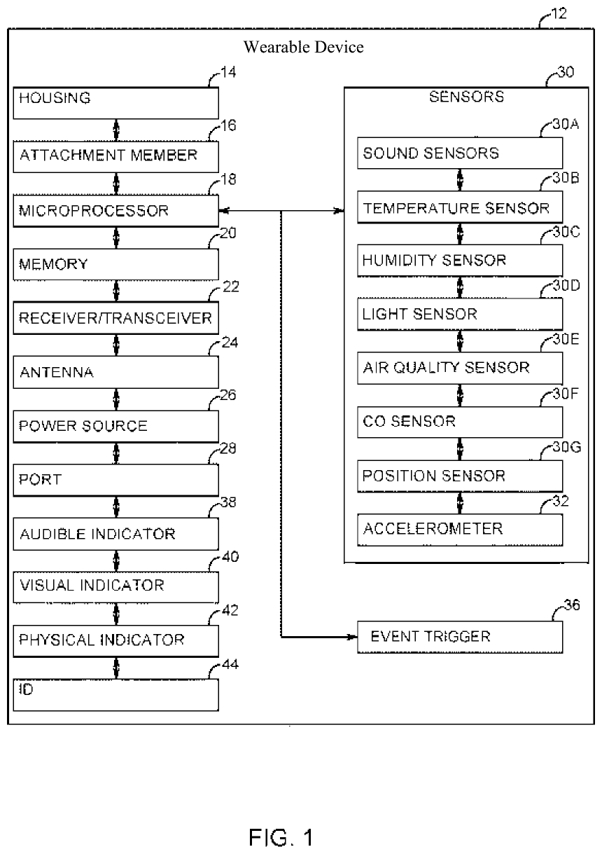

FIG. 1 shows a viewpoint of cameras and sensors capturing images and information from people. The client 110 is a smart phone including the cameras 112. Generally, most of smartphones support at least one front-facing and rear-facing cameras. The rear-facing cameras which are often used to take photos or videos may be higher resolution than the front-facing cameras. The method may be intended to simultaneously control at least one front-facing and rear-facing cameras of the client 110 capturing images or videos from people engaging in an event. The cameras 112 may support many types of lens such as telephoto, wide, ultra-wide, depth and other lens of specific feature. The view angle lines 120 of the cameras 112 may be determined by what kind of lens the client 110 uses. The lens of the client 110 may determine the angle of the view angle lines 120. The client 130 is a surveillance camera which supports networking functions, such as CCTV (Closed-Circuit Television) camera, IP (Internet Protocol) camera, IP dome camera and other cameras. The view angle lines 140 of the client 130 may depend on what kind of lens it uses.

The client 110 and 130 can capture images, videos or data of the users 150 and transmit the images or videos to the computing unit of the client 170, 180, local servers, remote servers or super computers for analyzing their behavior engaging in an event. Moreover, the client 110 may be a high end smartphone and take in charge of treating images, videos or data of the users 150 for analyzing their behavior of engaging in an event. The client 170 and 180 are a local server or personal computer and laptop, they may be including cameras which are also in charge of capturing the images or videos of the users 150. Basically, the client 110, 130, 170 and 180 may include any kind of communication unit supporting protocols 162 and 164, such as 4 or 5G (4 or 5 Generation), Wi-Fi, Bluetooth, NFC (Near-field communication) and other networking technologies. The client 160 may be a broad band device like router or other devices supporting 5G or 4G, Wi-Fi, fiber, Ethernet and other networking technologies. It may connect with the client 110, 130, 170 and 180 by using 5G or 4G, Wi-Fi or Ethernet networking technologies.

Eventually, the client 110, 130, 160, 170, 180, local servers, remote servers or super computers may be equipped with a variety of sensors to detect and collect a variety of information from user's engagement in an event. A diversity of events may happen in different venues. Different venues may be equipped with suitable clients or servers for specific purposes, it depends on their requirements. Currently, most of venues equipped with these devices don't provide any information of customers with other organizations or companies, the system as well. But, some of them may permit a contractor collecting information of user's behavior of engaging in an event from their clients or servers. The system may be intended to recruit them into a membership and configure their clients and servers for communication sessions. However, some venues may only provide proprietary services without equipping with any client or server. The system may be intended to skip them in user's healthcare plan. If a user insists to engage an event over there, the system may request a user replying information through their clients.

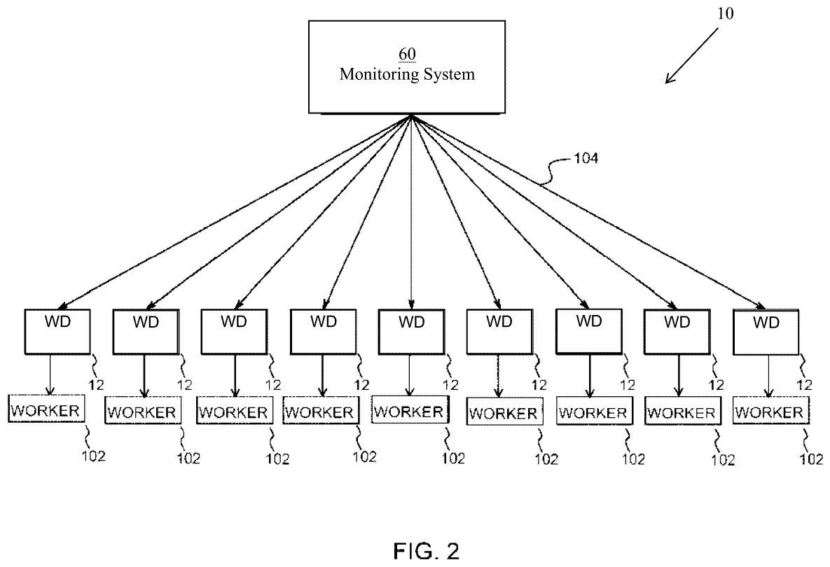

FIG. 2 is a block diagram of a system that may be used to implement the methods and systems described in the document. The system described herein includes server 210, network 220, smartphones 230, computers 240, peripheral smart devices 250 and peripheral devices 260. The server 210 may consist of multiple servers and connect with multiple clients via the network 220. In one example, other such entities may also exist in the system and be connected to the network 220. The server 210 may be located at local area or remote site and communicate with the multiple clients via the network 220. It may implement the method by using one or more servers and a cloud platform. The server 210 may be configured to implement the method providing personal health management, dietary management, nutrient's supplements management, exercise or activity arrangement and other services. It includes healthcare module 212, mathematics module 214, control module 216, membership module 218.

The functionality of the healthcare module 212, mathematics module 214, control module 216 and membership module 218 may be more sophisticated than the one of all clients' modules. So, all clients may be in charge of simple functionality, and transfer a plenty of data to the server 210 for computations and storage. The functionality of the healthcare module 212 may include healthcare plan establishment, knowledge and suggestion establishment, demonstration of process or event and process improvement. The functionality of the mathematics module 214 may include recognition of a variety of objects, evaluation of a variety of symptoms, calculations for information collected from user's engagement in an event and establishment of algorithm for optimization of user's healthcare plan. The functionality of the control module 216 may include establishment and management of multitasking operations for simultaneously treating processes, events, healthcare plans or multiple users, management of IOT, GPS applications and process monitoring. The functionality of the membership module 218 may include registration of membership, establishment of a diversity of databases, establishment of group for registered members and establishment of authorization between registered members.

The network 220 may be implemented by using standard protocols of communication such as Dynamic Host Configuration Protocol (DHCP), File Transfer Protocol (FTP), Hyper Text Transfer Protocol (HTTP), Internet Message Access Protocol (IMAP), Network News Transport Protocol (NNTP), Network Time Protocol (NTP), Post Office Protocol-Version 3 (POP3), Simple Mail Transfer Protocol (SMTP), Simple Network Management Protocol (SNMP), Multi-Protocol Label Switching (MPLS) and Transmission Control Protocol/Internet Protocol (TCP/IP). In one implementation, the 5th generation mobile networks (5G) may be one of suitable communication technologies. High bit-rate and low network-latency of the 5G network may make images and videos transferred smoothly between clients and servers.

The smart phones 230 is a computing device, which may include healthcare module 212, mathematics module 214, control module 216. Basically, the computing power of smart phone is less than one of the server 210. So, the healthcare module 212, mathematics module 214, control module 216 of smart phones 230 may be reduced models of the server 210 modules. These modules may be configured to execute a part of functions of the server 210 modules and transfer a plenty of data to local or remote servers via the network 220 for further computations. Generally, the smart phones 230 is a popular device for users to implement the method. They may initiate the applications by using the smart phones 230.

The system may be intended to establish communication sessions between the smart phones 230, local and remote server and other clients, and then make configurations for all the devices. The other clients mentioned herein include computers 240, peripheral smart devices 250 and peripheral devices 260. These devices may bundle a variety of sensors to detect or measure user's behavior or scenarios of processes they are engaging in. The system may be intended to monitor a user by using cameras, microphones, sensors and other components or modules of the smart phones 230, computers 240, peripheral smart devices 250 and peripheral devices 260. These clients may be configured to capture voice signals and images or videos of a user. In one implementation, during user's engagement in an event in a venue of a member, the system may be authorized to control the microphones and cameras of these clients to capture their voice and images or videos and transfer them to servers for analysis and computations. Otherwise, it may be authorized to only retrieve user's voice and images or videos from a storage of a client or server in a venue of a member.

The functionality of the healthcare module of 232, 242 and 252 may include a reduced version of healthcare plan establishment module, knowledge and suggestion establishment module, demonstration of process or event module and process improvement module. When a user wants to register a membership through these clients 230, 240 or 250, they may have to provide personal information, especially current health condition, to the system for a registration of membership. After a user finishes the registration of membership, these clients may be configured to instantly establish user's healthcare plan. Assuming the computation of the clients 230, 240 and 250 is normal, it may be configured to request the system establishing user's healthcare plan, and then receive the user's healthcare plan from the system in order to execute it for managing user's engagement in an event.

In one implementation, the system may be intended to track a user after they initiate applications of their healthcare plan or periodically request user's location information when they want to execute an upcoming event in their healthcare plan. It may know the venue in which they want to execute the event and what kind of devices the venue is equipped with. The system may be intended to distribute databases and tasks to these devices over there and assign one of these devices to be a master working together with the other devices in order to deal with user's engagement in an event. When a user wants to initiate an event of healthcare plan through the clients 230, 240 and 250, the healthcare module of 232, 242 and 252 may be installed with the related parts of functions of the healthcare module 212 including knowledge and suggestion, demonstration of process or event and process improvement from the system and store them into storages of these clients in order to locally treat problems produced by user's engagement in an event. It may prevent from operational mistakes and delays due to network latency.

Generally, the mathematics module of 234, 244 and 254 may also be installed with the related parts of functions of the mathematics module 214 including recognition of a variety of objects, evaluation of a variety of symptoms, calculations for information collected from user's engagement in an event and establishment of algorithm for optimization of user's healthcare plan. Moreover, the control module of 236, 246 and 256 may also be installed with the related parts of functions of the control module 216 including establishment and management of multitasking operations for simultaneously treating processes, events, healthcare plans or multiple users, management of IOT, GPS applications and process monitoring.

Most of peripheral devices 260 only including the control module 262 may be equipped with a simple computing board, cameras and specific sensors. The control module 262 may be configured to only transmit the collected information from user's engagement in an event to other smart devices for analysis and computations. It may support many communication protocols over short distances, such as Bluetooth, NFC (Near Field Communication), ZigBee, RFID (Radio Frequency Identification) and other communication technologies. Additionally, some stuffs in the area where a user locates, not including a simple computing board, may be put in the place where cameras cannot find them. This stuff, like a tag of RFID, is popular to be used in a diversity of applications. For example, the system may be intended to request user's client communicating with this stuff for confirming user's location in a venue or other purposes.

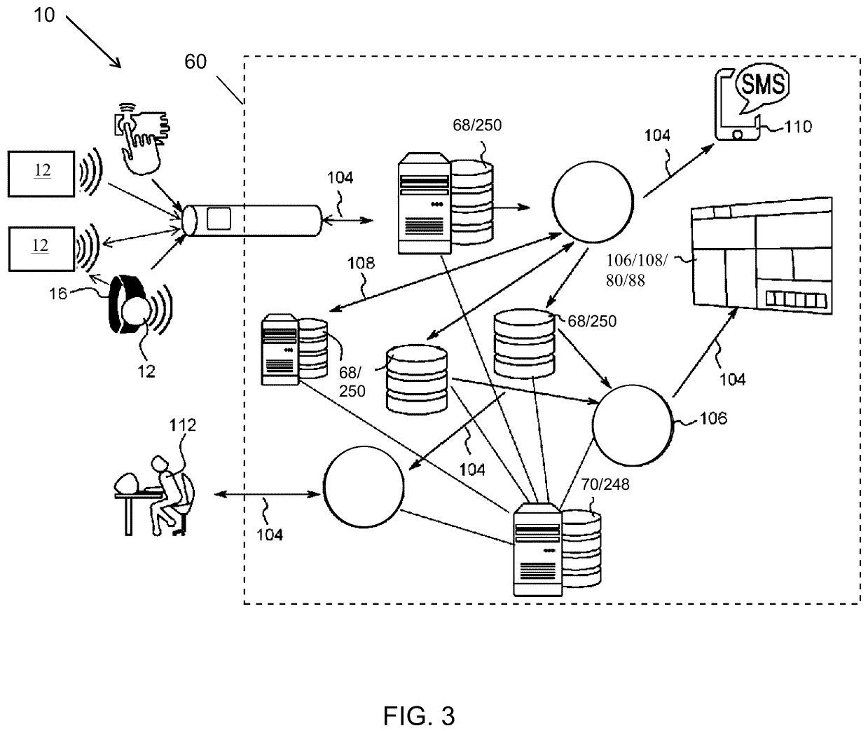

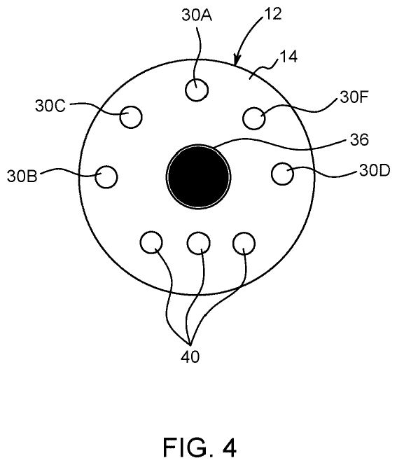

FIG. 3 is a block diagram illustrating the healthcare module of the server. The healthcare module 212 built in the server 210 includes healthcare plan establishment module 310, knowledge and suggestion module 320, demonstration module 330 and process improvement module 340. The healthcare plan establishment module 310 may be configured to build up a healthcare plan for a user based on their latest status after they register a membership. Their latest status includes weight, height, age, illness, BMI and other health information. It may periodically request health data of a user by using their medical instruments or inspection of a hospital. The healthcare plan mentioned herein is portfolios of healthcare plan, the portfolios include activity, exercise, diet, nutrients, assistance and other related plan.

In one implementation, the healthcare plan establishment module 310 may be configured to generate a variety of events according to the portfolios of healthcare plan for a period of time, and notify a user executing these events on time in order to make them healthy. An event may consist of a single process or variety of processes and contain a lot of predetermined information. The predetermined information mentioned herein may be nutrients, ingredients, calories, temperature of preserved or cooking, duration and consuming calories of exercise or activity and other related information. While a user is engaging in an event, the system determines user's symptom is getting worse, it may be intended to terminate the event and modify the portfolios of healthcare plan. Generally, a series of events in the portfolios of healthcare plan may happen in user's, family's or friend's house, their office, restaurants, supermarkets, shopping malls, gym, outdoor space etc. in a period of time. Except an event happens in user's house, the system may be hard to take an authorization for controlling a plurality of clients and sensors in the other places to collect information from user's behavior. But, it may be authorized to retrieve the information from the storage of their servers or clients.

The knowledge and suggestion module 320 is a module to establish a diversity of knowledge, such as nutrition, medical treatment, symptoms diagnosis, disease, exercise, diet, cuisine and other related knowledge, and suggestions for making a judgement by a user while they are engaging in an event. It may be configured to periodically search for the latest knowledge on the internet or arranged by a service's provider or system developer. It is a database to provide information to a user or other modules in the system for adjustment, reference, comparison and other purposes. While a user is engaging in an event and close to a predetermined area enclosing venues of dietary services or activities, the GPS module 530 may be configured to form a list of dietary services or activities suitable to carry out the portfolios of the healthcare plan in the period of time based on the location information of the dietary services or activities.

In one implementation, the knowledge and suggestion module 320 may be configured to illustrate suggestions for choosing the dietary services or activities in the list to the user. After the user makes a choice for the dietary services or activities in the list based on the suggestions, the system may be intended to monitor a user engaging in a series of procedures of an event. When it finds out a user hesitates or makes wrongdoing during the engagement in the event, it may provide demonstrations or suggestions of how to complete the event. For example, a user plans to cook a meal at home. The method may be intended to initiate a plurality of clients and sensors nearby the user for monitoring their behavior and demonstrating the cuisine of the meal before the event starts or during the cooking in order to prevent from a wrongdoing or incident.

The demonstration module 330 is a module to establish a communication session for connecting clients and sensors involving in an event in an area where a user locates in order to demonstrate processes of the event to a user when they hesitate or make a wrongdoing during an engagement in the event. Assuming a user makes a wrongdoing, the method may be intended to first interrupt the event and request the healthcare plan establishment module 310 providing all processes of the event to the demonstration module 330. The demonstration module 330 may be configured to control clients and sensors involving in the event in the area where the user locates for demonstrating part or all of processes of the event to the user. In one implementation, it may be configured to demonstrate all procedures of the event for teaching the user how to complete the event when the event is initiated and the user decides to do it. Moreover, it may be configured to illustrate user's health status via a client carried by or nearby the user before they execute the event.

The process improvement module 340 is a module to find out an improper process in the healthcare plan and modify it. Basically, an event consists of many processes, an amount of processes in an event depends on user's requirement. A result of each process may be predetermined by the system. In one implementation, during user's engagement in an event, the system may be intended to compare a result of user's engagement with the predetermined one each process. Assuming the result of user's engagement each process is different from the predetermined one, it indicates that a deviation happens. When a deviation causing a negative result of user's engagement in the event happens, it indicates the result is deviated from the purpose of the event and may make user's health worse. The system may be intended to first terminate the user's engagement in the event and figure out which part of processes in the event the deviation happens, then analyze what causes the deviation in order to modify the user's healthcare plan. At the same time, it may suggest the user stops this event and continue next event.

FIG. 4 is a block diagram illustrating the mathematics module of the server. The mathematics module 214 built in the server 210 includes object recognition module 410, symptom evaluation module 420, calculation module 430, algorithm module 440. The object recognition module 410 is a module to recognize an object, including image, oral, voice, data etc., collected from user's behavior and appearance by comparing it with the one in the database of the knowledge and suggestion module 320. The operations of recognition for an object may include a diversity of computations for the object collected from user's behavior and appearance to transform it into a variety of values for comparisons with the predetermined one in the database. The system may be intended to establish a list for a variety of categories of predetermined objects for comparisons with the object collected from user's behavior and appearance during their engagement in an event.

When it gets an object transferred from the process monitoring module 540, it may analyze features of the object in order to figure out their category, then inform the database establishment module 620 to retrieve the predetermined object in the databases for further analysis. In one implementation, the process monitoring module 540 may be configured to periodically control cameras and sensors nearby a user engaging an event to capture images and related data from user's appearance, body status and behavior, and transfer them to the object recognition module 410. The object recognition module 410 may be intended to analyze the images and related data to get their categories and transfer them to the database establishment module 620 for retrieving predetermined images and data for further recognition. Assuming it determines the category is belong to symptoms, it may be configured to transfer them to the symptom evaluation module 420 for further analysis.

The symptom evaluation module 420 is a module to judge user's symptom is getting better or worse while they are engaging an event. It may be configured to establish a list for a diversity of categories of predetermined symptoms for comparison. The predetermined symptoms mentioned herein may include images and data of symptoms collected from the internet or prearranged by system or service providers. When the object recognition module 410 gets an object transmitted from the process monitoring module 540, it may analyze features of the object in order to figure out their category, then directly retrieve predetermined object in databases or inform the database establishment module 620 doing these operations for further analysis. In one implementation, the process monitoring module 540 may be configured to periodically control cameras and sensors nearby a user engaging an event to capture images and related data from user's appearance, body status and behavior, and first transfer them to the object recognition module 410. Assuming it determines the category is belong to symptoms, it may be configured to transfer them to the symptom evaluation module 420 for further analysis. The symptom evaluation module 420 may be configured to analyze the images and related data to get their category and transfer them to the database establishment module 620 for retrieving predetermined images and data for further recognition.

The calculation module 430 is a module to make calculations for user's health status and information collected from a plurality of clients and sensors nearby a user during an engagement of their event. Categories of information may include user's health data, serving, calories and nutrients of food or drinks, exercise or training, harmful substances, etc. The method may be intended to transform the information collected from a plurality of clients and sensors nearby the user during the engagement of their event to a lot of values and make calculations for the values and data of user's health. The results of the calculations may be configured to provide to other modules or a user to make a lot of judgements for further treatment or subsequent operations.

The algorithm module 440 is a module to establish an algorithm for optimizing processes of an event and portfolios of healthcare plan. It may be configured to dynamically rearrange an order of processes of an event or portfolios of healthcare plan during user's engagement in the event. It may also be configured to dynamically modify scenarios of processes of an event and portfolios of healthcare plan during user's engagement in the event. The method may be intended to analyze information collected from a plurality of clients and sensors nearby a user during user's engagement in an event in order to rearrange next processes or events, or modify scenarios of processes or portfolios of healthcare plan. When a deviation causing a negative result of user's engagement in an event happens, it indicates the result is deviated from the target of the event and may make user's health status worse. The algorithm module 440 may be configured to optimize part or all of processes of an event causing the deviation for preventing from the deviation happening again. The method may be intended to modify the user's healthcare plan based on the optimization on the processes of the event.

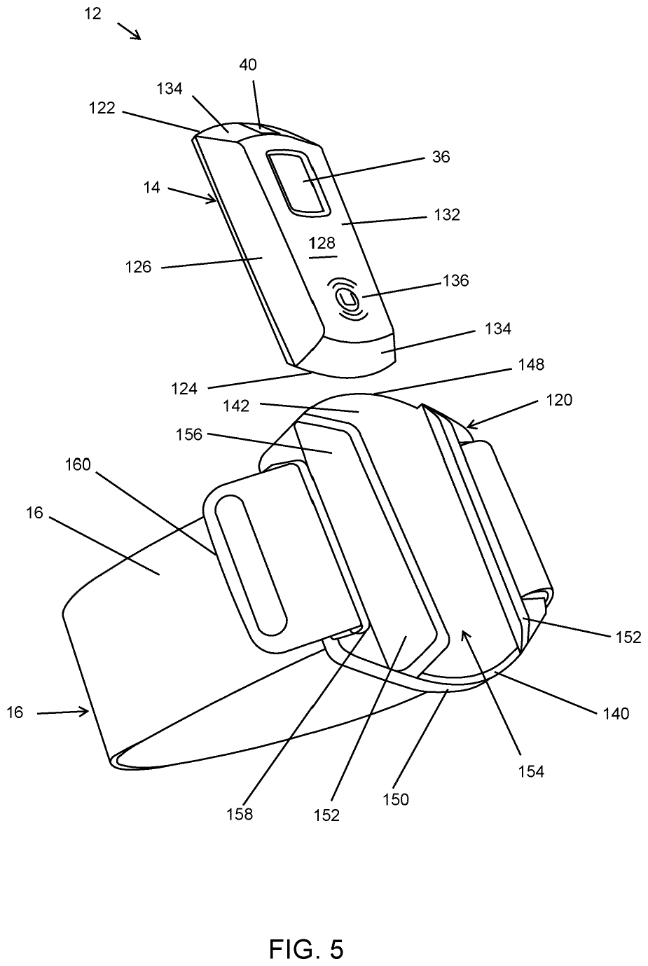

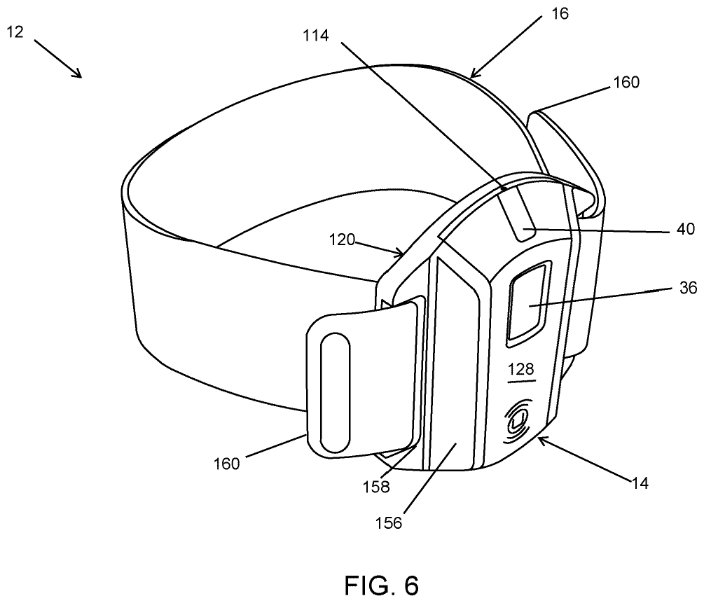

FIG. 5 is a block diagram illustrating the control module of the server. The control module 216 built in the server 210 includes multitasking management module 510, IOT management module 520, GPS module 530 and process monitoring module 540. The multitasking management module 510 is an intelligent module to arrange multitasking operations for treating a lot of tasks happening simultaneously and treat a diversity of sophisticated operations between other modules. It may simultaneously receive a lot of requests from other modules and transform these requests to many tasks, then distribute the tasks to other modules for further operations.

Additionally, it may be configured to establish a lookup table for recording their connections and relationships for these requests and tasks. It may periodically monitor these modules processing their tasks assigned by the multitasking management module 510. In one implementation, the healthcare plan establishment module 310 may be configured to set up a variety of events for portfolios of healthcare plan for a user in a period of time, then transfer the variety of events and portfolios of healthcare plan to the multitasking management module 510. The multitasking management module 510 may be configured to execute the portfolios of healthcare plan in the period of time and request the GPS module 530 periodically tracking movements of the user based on their location information.

The GPS module 530 may be configured to navigate them to the place or notify a venue of service's provider or individual is close to them for executing an event over there. It may trigger an event initiating a plurality of clients and sensors in the place or venue for collecting a lot of information from user's behavior. When the user decides to execute an event in the venue of service's provider or individual, the multitasking management module 510 may be configured to arrange all procedures of the event in the early stage of the event and command the IOT management module 520 setting up communication sessions between sensors, clients and servers in the venue of service's providers or individual in other to make the system or user's client collected a predetermined information of product, exercise or activity. After it gets the information from the IOT management module 520, it may command the calculation module 430 making calculations for the user's health status and the predetermined information in the early stage of the event, then make estimations and suggestions based on the calculations of the user's health status and predetermined information for the user. The user may be requested to make a decision about whether they engage in the event or not.

In one implementation, assuming they decide to execute the event, the multitasking management module 510 may be intended to command the demonstration module 330 to demonstrate all of processes of the event by means of the plurality of clients and sensors in the venue for instructing them how to complete the event in the early stage of the event. When the user starts engaging in the event, the multitasking management module 510 may be intended to command the process monitoring module 540 to monitor and collect information from the user engaging in the event by means of the plurality of clients and sensors. After the collection of the information, it may be intended to command the object recognition module 410 to analyze the information for classifying their categories. The categories mentioned herein include symptom, calorie, ingredient, user's health data, nutrients, action of exercise etc.

The object recognition module 410 may be requested sending the categories back to the multitasking management module 510. Basically, the multitasking management module 510 may be intended to dispatch these objects to the proprietary modules for treatments according to the categories, then command the calculation module 430 making a variety of calculations for the user's health status and collected information. Assuming the user reaches the target of the event before the event finishes, the multitasking management module 510 may intentionally interrupt the user engaging in the event and illustrate the updated status of their health.

Otherwise, it may be intended to continuously monitor the user's engagement until the event finishes. In one implementation, it may be intended to initiate a plurality of clients and sensors, especially instruments, involved in the event in the area where the user locates or request the user using instruments without a networking function to measure the user's health status when the event is completed. It may be intended to request the user replying the measuring result by their client, and then update the user's health status and portfolios of the plan in response to the measuring result for the user's health status.

The IOT management module 520 is a module to set up communication sessions for registered clients and sensors nearby a user engaging an event for capturing images and related data from user's appearance, body status and behavior. It may be configured to transmit the captured images and related data to the object recognition module 410 for further analysis. In one implementation, the system may periodically request location information of a user and transmit the location information to the server 210. It may be intended to track movements of the user for determining when an event is coming.

When it detects the user arrives at the venue suitable to executing the event by comparing location information of the user and venue, the IOT management module 520 may set up communication sessions for registered clients and sensors in the venue where the user engages in the event for capturing images and related data from user's appearance, body status and behavior, and then transfer them to the object recognition module 410 for further analysis. However, most of service providers may not permit the system directly controlling their clients and sensors to capture the images and related data from user's appearance, body status and behavior. But, it may allow the system to retrieve the images and related data from storages of their server or intelligent client.

The GPS module 530 is a module to navigate a user to a venue of service's providers or private for executing an event over there. It may be configured to form a list for venues of dietary services or activities suitable to carry out the event's purpose in the period of time by determining the user is close to a predetermined area enclosing the venues of the dietary services or activities. The predetermined area described herein may be prearranged in the predetermined shape, for example circle, triangle, rectangle, square etc., to cover all positions of venues of service providers and preset by a system designer.

Additionally, the system may be intended to preset the predetermined area to enclose a lot of venues of registered service providers based on the portfolios of the user's healthcare plan. So, it may be established based on the current location information of the venues of the dietary services or activities. When the user moves towards the predetermined area and confirms one of events, the system may initiate the event activating servers or a plurality of clients and sensors in the venue for collecting a lot of information from user's behavior. In one implementation, the GPS module 530 may be configured to activate GPS functions of user's client in response to the user leaving the venue and deactivate the GPS functions when the user arrives at the venue for engaging in the event. The user's client may be configured to periodically transmit their location information to the server 210.

The GPS module 530 may be configured to periodically collect location information of a client according to predetermined frequency values. The predetermined frequency values described herein are used to determine frequencies of receiving location information from the GPS satellites and transmitting the location information to the server 210 respectively by user's client. The predetermined frequency values may be configured by a system designer. The server 210 may configure two different timers for receiving their location information and transferring their position information to the GPS module 530. Eventually, a GPS module of a client may frequently miss GPS signals from the GPS satellites and be unable to form a position of the client. The GPS module of the client may transmit the previous position instead of the current one to the GPS module 530.

The process monitoring module 540 is a module to set up communication sessions for connecting clients and sensors involving in an event in an area where a user locates in order to monitor the user engaging in processes of the event. It may be configured to find out that they make a wrongdoing or their symptoms is getting worse during their engagement in the event. When they make a wrongdoing or their symptoms is getting worse during their engagement in the event, the method may be intended to first interrupt their engagement in the event, and then request the demonstration module 330 or algorithm module 440 to demonstrate or modify part or all of processes of the event.

While a user is engaging in an event, the process monitoring module 540 detects the plurality of clients and sensors unable to collect information from the user's behavior, it may interrupt the engagement of the user for illustrating which part of the event the plurality of clients and sensors are unable to collect the information from. Then, it may request the user to reply the information by speech, words etc. in order to continue the user's engagement in the event. In one implementation, it may detect user's health status deviates from a predetermined information of an event based on a series of information collected from user's behavior while a user is engaging in the event. The method may notify the user about the deviation from the predetermined information of the event in order to interrupt their process. It may be intended to perform a demonstration or termination of procedures of the event based on a result of analyzing the deviation. Assuming the deviation causes a negative result, it may be intended to modify the part or all of processes of the event or portfolios of the user's healthcare plan in the period of time.

FIG. 6 is a block diagram illustrating the membership module of the server. The membership module 218 built in the server 210 includes registration module 610, database establishment module 620, group establishment module 630 and authorization module 640. The registration module 610 is a module to allow a user or organization to make a registration of a membership. It may be configured to request a user to make a registration for themselves, their servers, clients and sensors in their venue, additional users living or working there as well. The venue mentioned herein includes user's home or organization's office or building. The organization includes a variety of supermarkets, shopping malls, stores, gyms, hospitals, government's bureaus etc. After the registration of membership, the system may be permitted to directly access storages of their server or client for retrieving a lot of information collected from user's behavior of engaging in an event. It may also be authorized to directly control their clients for more operations.

The database establishment module 620 is a module to establish a diversity of databases of knowledge, technologies, symptoms, user's health status, descriptions of products, calories consumed by exercise or activity and other information. These databases include portfolios of nutrients from food and drinks, cuisine, exercise, medical diagnosis, portfolios of symptoms and other applications. It may be configured to periodically update these databases for the latest information. The portfolios of nutrients from food and drinks mentioned herein include a diversity of vitamins, minerals and other materials. It may intentionally be arranged based on user's health status.

The databases of cuisine described herein includes way, time and order of cooking, material, calories and other related knowledge. The databases of exercise include a variety of knowledge about activities, such as hiking, climbing, sports, swimming and others, training portfolios and consuming calories. The medical diagnosis is a technique to analyze a diversity of diseases based on a variety of symptoms of sickness and user's behavior. The ingredients and calories of food and drinks are provided by manufacturers, supermarkets, stores, restaurants and others. Moreover, the database establishment module 620 may be configured to periodically request a registered user updating their latest health status or providing a report of hospital inspection. It may also request service providers to update the predetermined information of their services or products, such as ingredients and calories of products, calories consumed by exercise or activity and other related information.

The group establishment module 630 is a module to permit a member establishing a new group for specific purposes. The specific purposes mentioned herein include individual, mutual or organizational assistant, prevention from incident happening and others. It may permit a registered member or organization to create a new group for their requirements. After a new group establishes, they can recruit additional users or organizations into the new group and then make a lot of operations for each other. In one implementation, a user or organization can be recruited into a diversity of groups at the same time in order to make a variety of interactive operations.

The supervisor establishment module 640 is a module to establish a supervisor to monitor and assist other user or organization in a group. It may build up a supervisory rule and priority for managing all of users or organizations in a group. The supervisor and priority may be arranged by the system or determined by all users or organizations. In one implementation, the supervisor may be intended to periodically monitor other users or organizations in order to get information about their status for analyzing the information. When an incident happens to a user or organization, the supervisor may immediately treat it or request additional users or organizations to assist the user or organizations, especially one nearby.

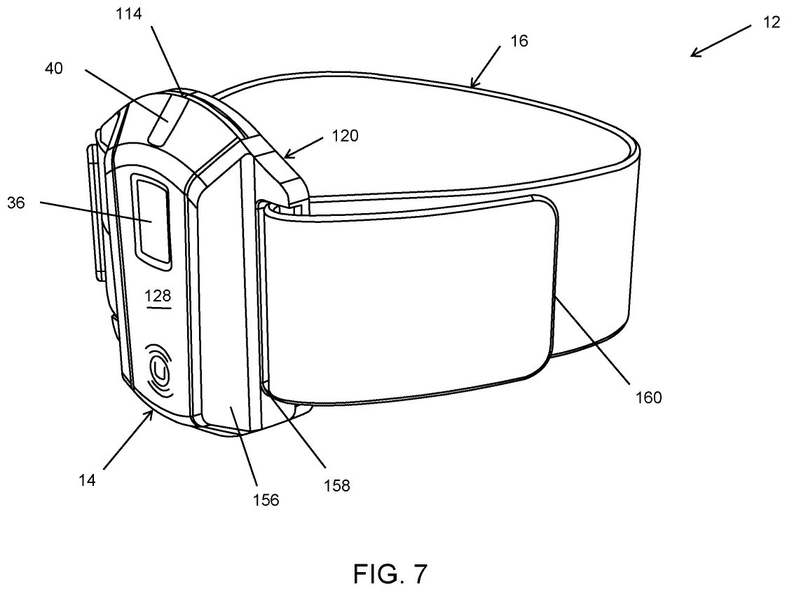

FIG. 7 is a flowchart of a process for implementing healthcare method. At box 710, illustrate user's health status and healthcare plan when a user starts an event and collect a predetermined information of the event. The box 710 represents that the system detects a user starts an event in portfolios of their healthcare plan, then illustrates the user's health status and healthcare plan and collects a predetermined information of the event. The portfolios of the healthcare plan mentioned herein are a combination of a diversity of plans good for user's health. The method may be intended to collect and diagnose user's health status, arrange portfolios of healthcare plan for a period of time, monitor their engagement in the portfolios of healthcare plan and health status and modify portfolios of healthcare plan when detecting the user's health status is getting worse. The portfolios of healthcare plan may consist of a variety of events like activity, exercise, dietary service, purchase for food and drinks, cuisine, supplement of nutrients etc. Most of the events may be requested to provide the predetermined information of the event in advance in order to provide the estimation of the event to a user.

At box 712, provide a suggestion based on the estimation of the data of the user's status and predetermined information. The box 712 represents that the system may be intended to make the estimation of the user's status and predetermined information in an early stage of an event for providing a suggestion to a user. Basically, the portfolios of user's healthcare plan may be prearranged by the system and include a diversity of events for a period of time which the user is requested to carry out. The system may be intended to periodically request user's location information to make an illustration for prearranged events which the user can choose, and further navigate them to the venue. In one implementation, when the user arrives at the venue in which a variety of events or a lot of choices can be executed or chose for an event, the method may make many estimations based on the variety of events or choices to provide suggestions to the user. It may be intended to make a variety of operations of mathematics to get an estimation by using the user's health status and predetermined information.

At box 714, demonstrate entire process of the event after confirming a decision of the user. The box 714 represents that the system may be intended to confirm the decision for a variety of events and choices with the user, and then demonstrate entire process of the event which the user chooses. Eventually, a venue or event in a predetermined area may have a lot of choices which a user can choose at the same time. When the user moves into a predetermined area where a variety of events and choices can be executed by them, they may be allowed to engage in many events and choices in the predetermined area at the same time based on the suggestions. The system may be intended to confirm their decision for these events and choices, then arrange demonstrations for the entire processes of the events or choices. Assuming they simultaneously choose many events or choices in the predetermined area, the method may be intended to suggest an order to execute the events or choices. It may demonstrate the processes of the events or choices one by one based on the order.

At box 716, analyze the lot of data collected from the user's behavior of engaging in the event. The box 716 represents that the system may be intended to analyze the lot of data collected from the user's behavior of engaging in the event. The method may be intended to classify the lot of data collected from the user's behavior into several categories and transmit them to proprietary modules in the system for further operations. The categories described herein is like symptom, data of measuring user's body values, weight of food, drinks, ingredients and other materials, procedures of cuisines, exercise and activities, etc. The proprietary modules may be configured to analyze the individual data distributed from the multitasking management module 510, and send the results back to the multitasking management module 510 after the analysis of the individual data.

At box 718, request the user replying information when detecting the information can't be collected from the user's behavior. The box 718 represents that the system may be intended to request the user replying information when it detects the information can't be collected from the user's behavior. Basically, the system may be hard to get exact information of something when it can not capture the information through a plurality of cameras or sensors nearby a user. For example, a user is preparing materials to cook their meal without weighing the materials. Most of users cook their meal by means of their experience without calculating weight or portion of materials. So, the system can't get exact data about the weight or portion of materials. Moreover, the duration of cooking may change an amount of nutrients in the food. Cooking too long may damage and reduce nutrients in the food, cooking temperature as well.

At box 720, makes calculations for a lot of data and the user's status. The box 720 represents that the method may be intended to makes calculations for the lot of data and user's health status by using a variety of mathematical operations. The system may be intended to monitor the user's health status during the engagement in the event by means of a variety of mathematical operations for the lot of data and user's health status. In one implementation, the method may be intended to capture information from user's behavior of engaging an event, and simultaneously make a lot of mathematical operations for the information and user's health status in the time-division multiplexing technology in order to instantly detect the deviation of user's engagement in the event. The time-division multiplexing technology described herein may be defined by a system developer.

At box 722, demonstrate procedures of the event when the deviation is caused by user's wrongdoing. The box 722 represents that when the system detects the deviation of a result of user's engagement caused by user's wrongdoing, it may be intended to arrange the demonstration for part or all of procedures of the event. The system may be intended to capture information from user's engagement in an event through a plurality of cameras and sensors nearby a user, and judge whether the result of the user's engagement in the event is getting better or worse by analyzing the information. Basically, the wrongdoings of user's engagement in an event may be figured out from user's behavior dealing with the processes of the event. Assuming the deviation is caused by user's wrongdoing, the system may be intended to stop the user's engagement to instruct them correcting the wrongdoing by demonstrating part or all of procedures of the event. After the correction on the wrongdoing, it may intentionally notify the user continuing their engagement in the event.

At box 724, terminate the event and modify the plan when the deviation is caused by improper processes in the event. The box 724 represents that when the system detects the deviation is caused by improper processes in the event, it may be intended to terminate the user's engagement in the event and modify the user's healthcare plan. Basically, the system may be intended to monitor the result of user's engagement in the event through a plurality of cameras and sensors nearby the user. When the deviation causing a negative result of user's engagement in the event happens, it indicates the result is deviated from the target of the event and may make user's health status worse. The system may be intended to first terminate the user's engagement in the event and figure out which part of processes of the event the deviation happens, then analyze what causes the deviation in order to modify the user's healthcare plan. At the same time, it may suggest the user stops this event and continue next event.

At box 726, does the event reach a target, finish or terminate? The box 726 represents that the system is intended to confirm a status of an event a user engages to be reaching a target, completion or termination. If the status is reaching a target, completion or termination, the system may be intended to jump to run the box 728. Otherwise, it may be intended to jump to run the box 716.

At box 728, update the user's status and initiate next event. The box 728 represents that the system may be intended to update the user's health status and then initiate next event after the status of the event the user engages is confirmed to be reaching a target, completion or termination. If the user reaches the target of the event before the end of the event or complete the event, the system may be intended to update the user's health status based on the results of all calculations for information collected from user's behavior of engaging in the event. Regarding the event reaching the target before the end of the event, the system may be intended to modify the processes of the event based on it to be a new event and include the new event into the user's healthcare plan. If the event is terminated, the system may be intended to find out improper processes in the event, and modify it to be a new event or remove it. Then, it may be intended to update the user's healthcare plan and initiate next event.

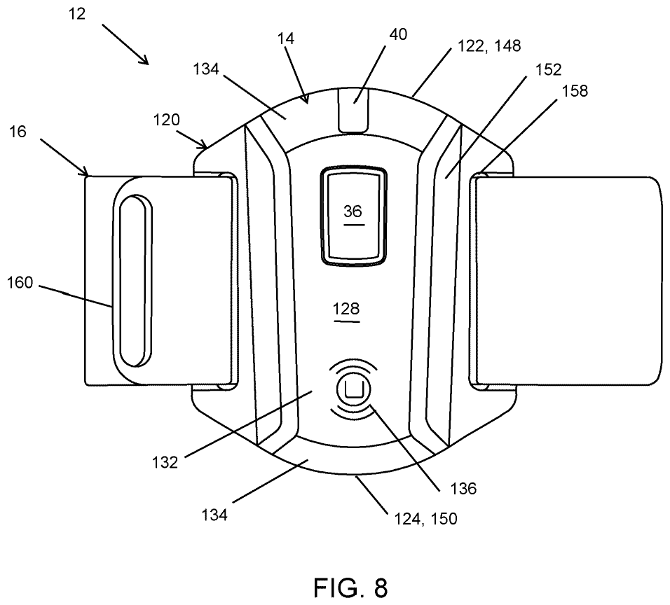

FIG. 8 is a block diagram illustrating components of a computing device that may be used to implement the methods and systems described in the document, as either a client or server. The computing system 800 represents variety kinds of computing devices such as desktop, notebook, workstation, server, mainframe, and other similar computing devices. The computing system 800 described herein may be represented as the server 210, the client 240 and 250. The computing system 800 includes CPU (Central Processing Unit) 810, graphics 820, I/O devices (Input/Output) 830, an audio 840, a peripheral control hub 850, memories 860, storage devices 870 and a network 880.

In one implementation, the CPU 810 may consist of multiple CPU chips and interconnect together by using dedicated high-speed interfaces. Each CPU chip has specific high-speed interfaces connecting to graphics 820 and memories 860. The CPU 810 may include a memory controller to interact with the memories 860. The memories 860 may consist of multiple DRAM (Dynamic Random Access Memory) chips and/or modules such as DDR4 SDRAM (Double-Data-Rate Fourth Generation Synchronous Dynamic Random Access Memory) chip and other DRAM chip. In another implementation, the CPU 810 may build in a non-transitory computer-readable medium such as NOR Flash (NOR Gate Flash) for storing a set of instructions.

The peripheral control hub 850 is a processor to interact with a plurality of peripheral devices by using specific low-speed interfaces, and connects with I/O devices 830, audio 840, storage devices 870, network 880 and additional peripheral devices. The I/O devices 830 is input or output devices (e.g., mouse, keyboard, USB Hub or LED) used to input data to the peripheral control hub 850 or output data by using specific devices. The audio 840 may include an audio codec chip for encoding or decoding audio data streams from or to the peripheral control hub 850 and connect with variety sound devices (e.g., speaker or microphone).

The storage devices 870 are non-transitory computer-readable media such as HDD (Hard Disk Drive) and/or SSD (Solid State Drive) for storing variety computer programs, apps, photos, videos and other information. In one implementation, the storage devices 870 can be internally, locally or remotely connected to the peripheral control hub 850 by using internal interfaces, SAN (Storage Area Network) or NAS (Network Attached Storage) protocol. The system is intended to execute specific operations, computer programs and/or apps may be loaded from the storage devices 870 to the memories 860. The network 880 can support variety technologies, such as Ethernet, Wi-Fi, fiber, and 5G, to connect with the network 220.

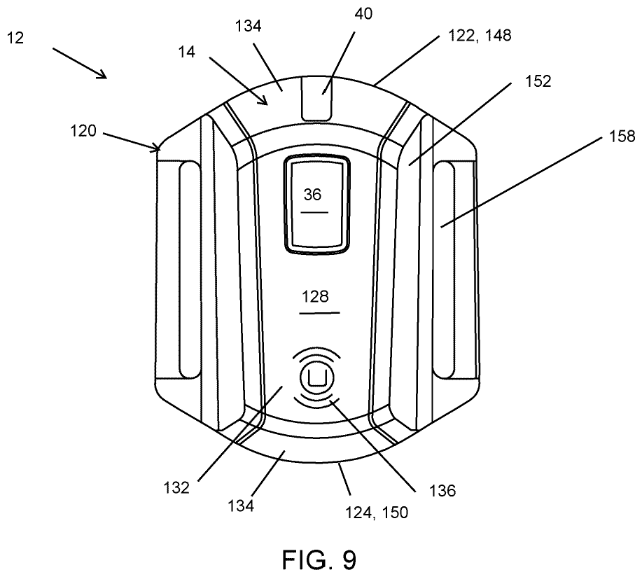

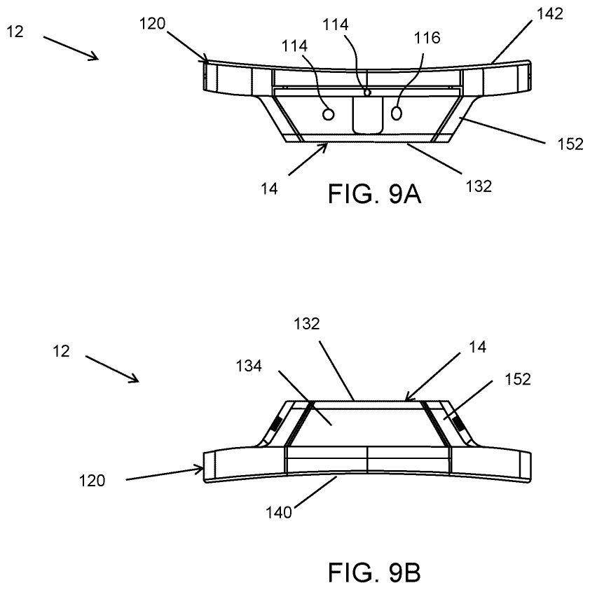

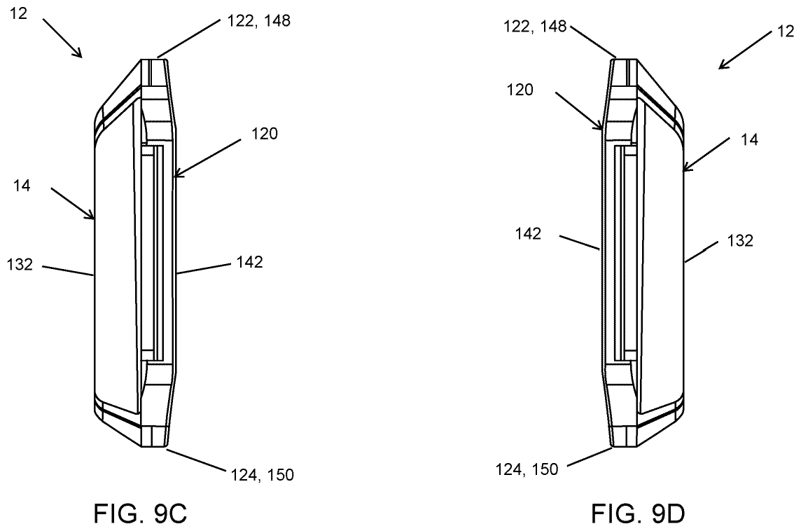

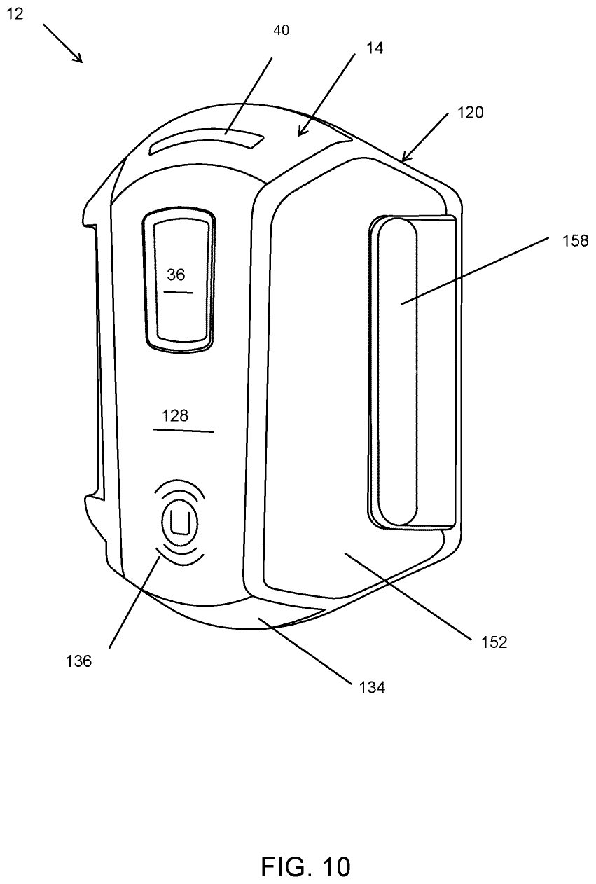

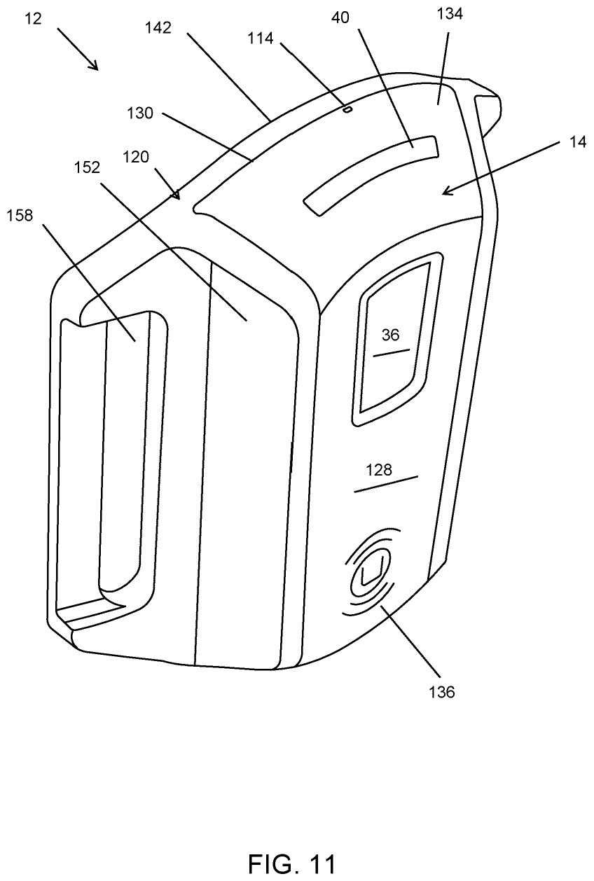

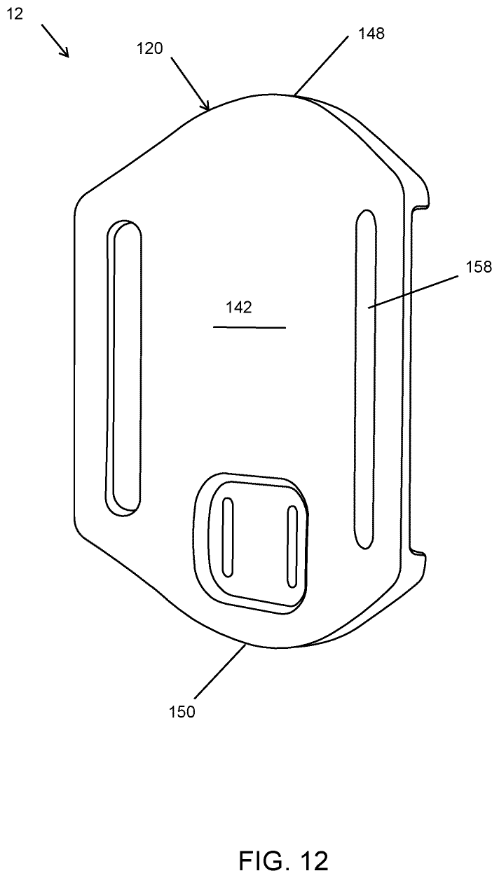

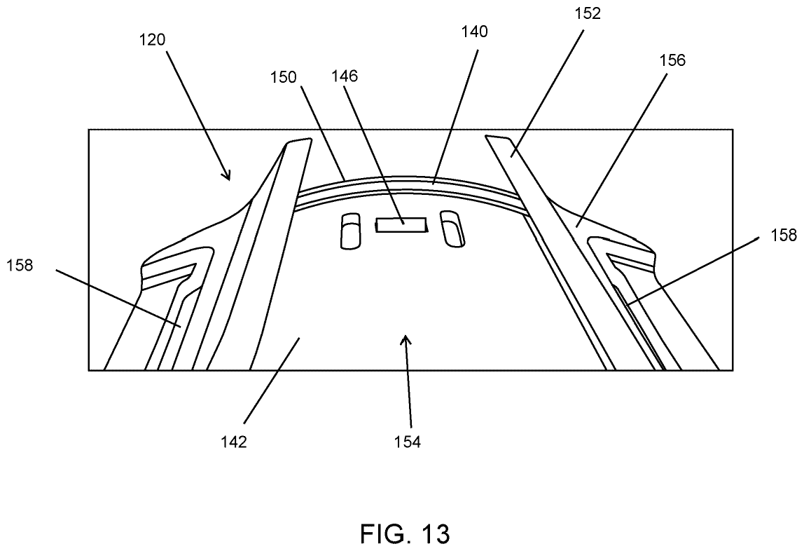

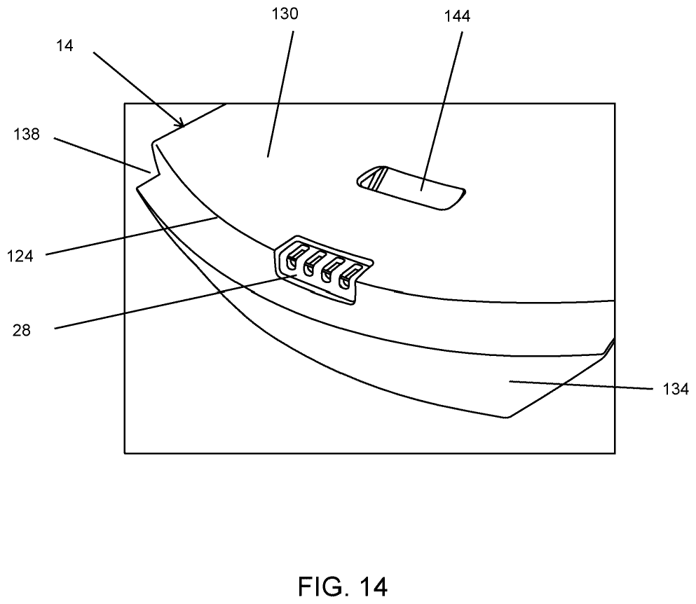

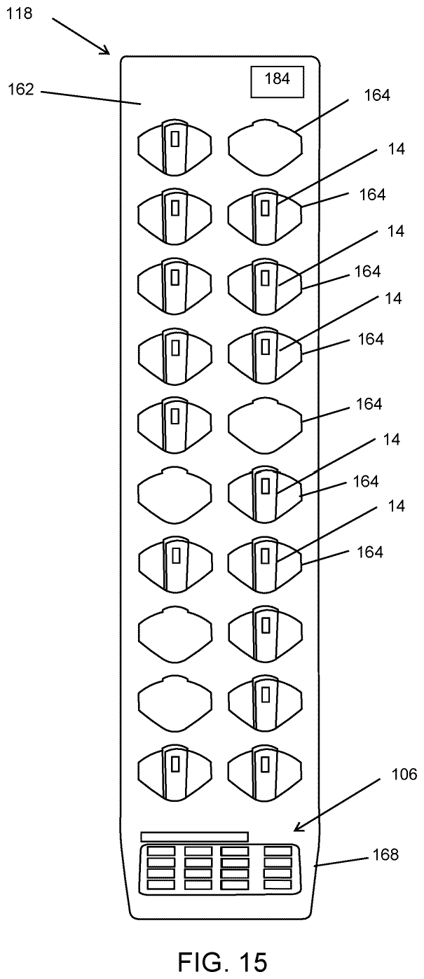

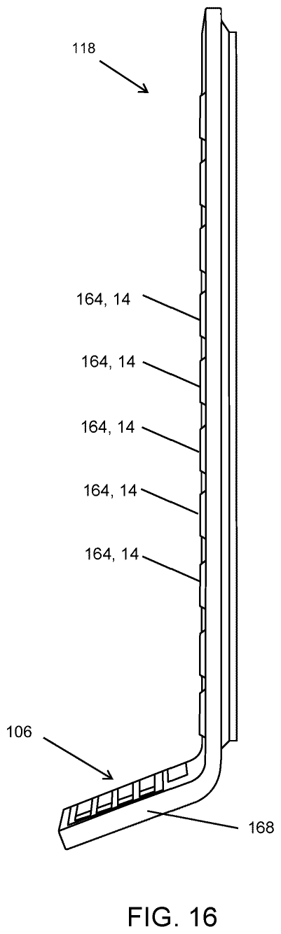

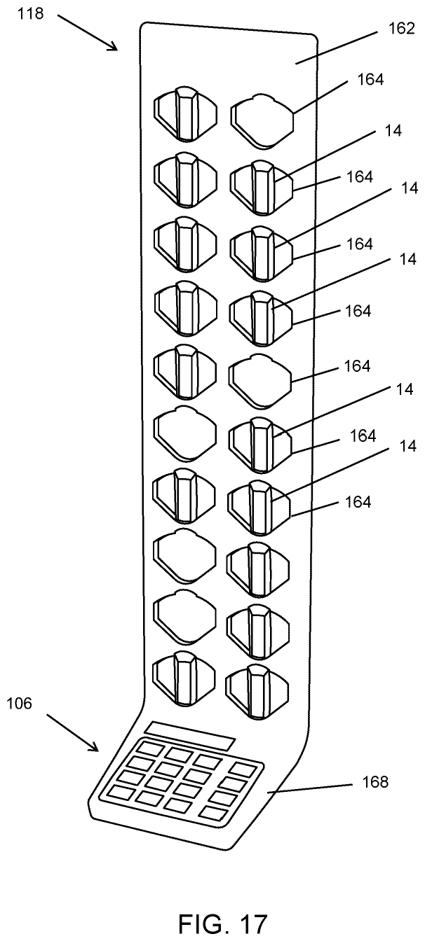

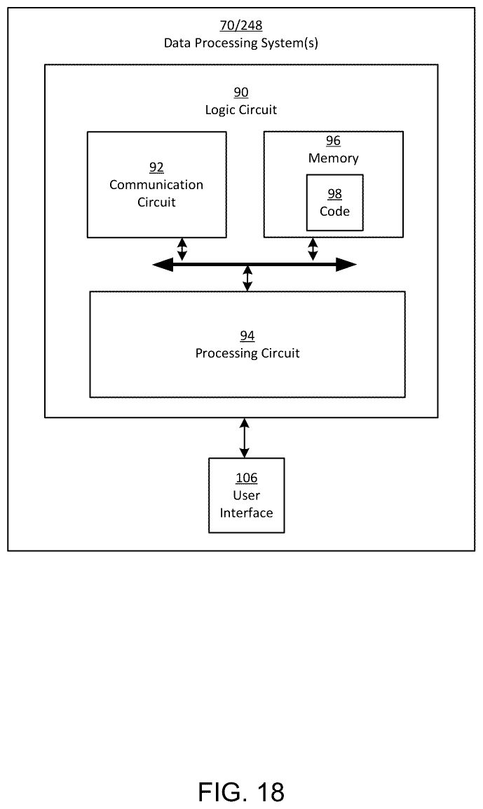

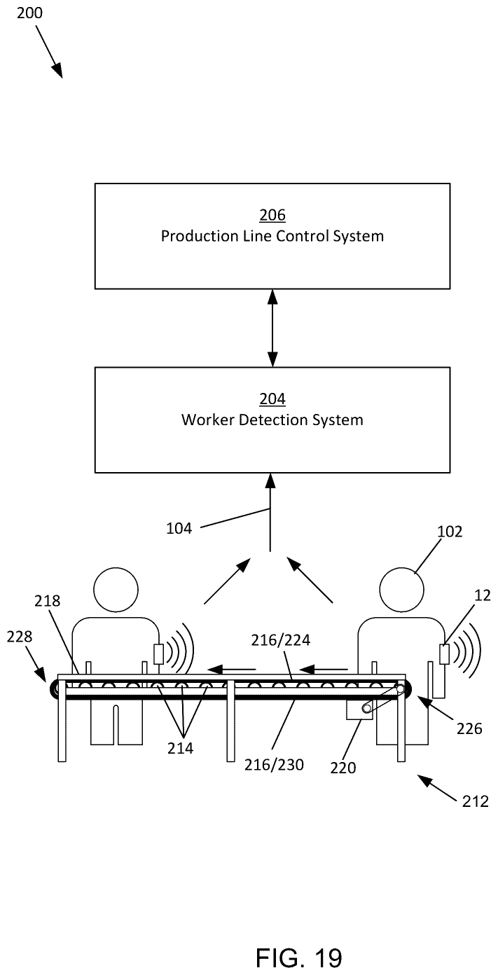

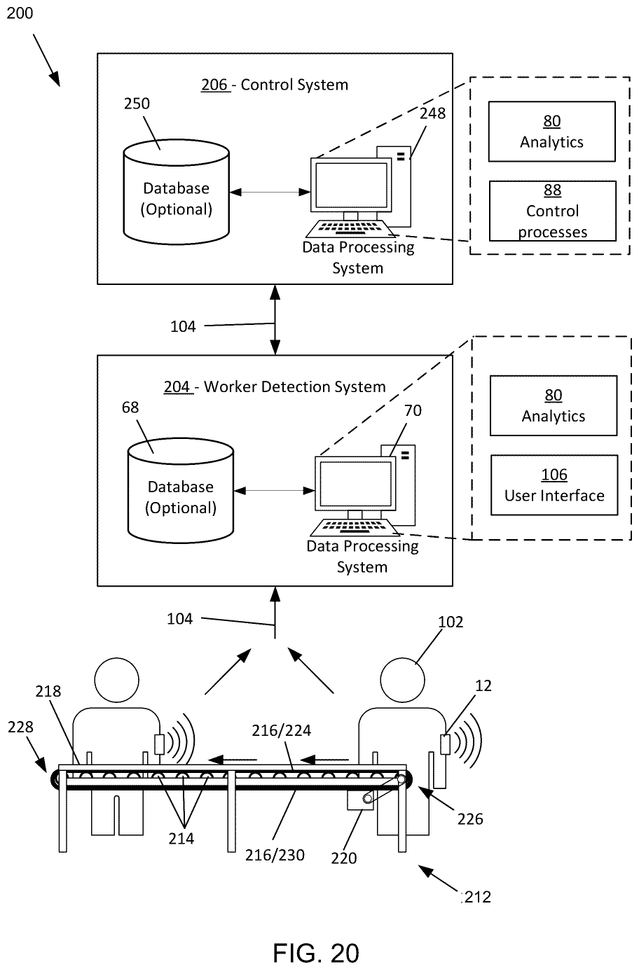

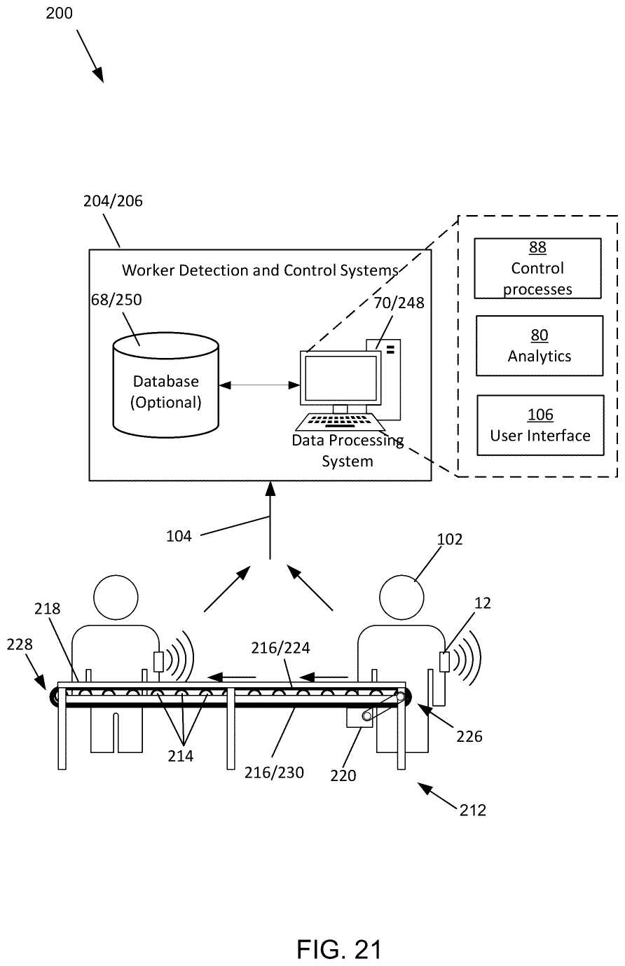

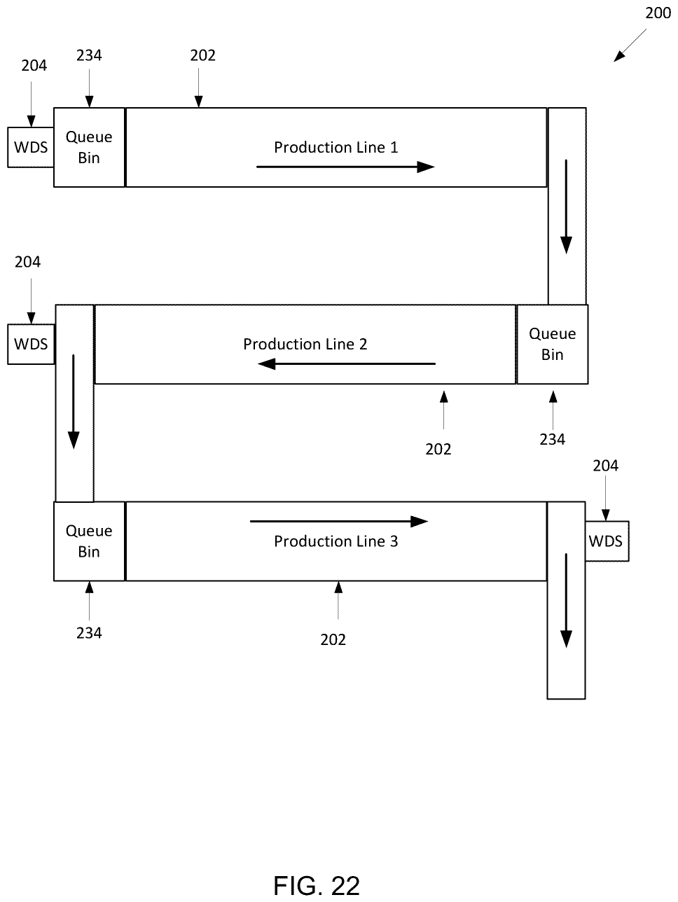

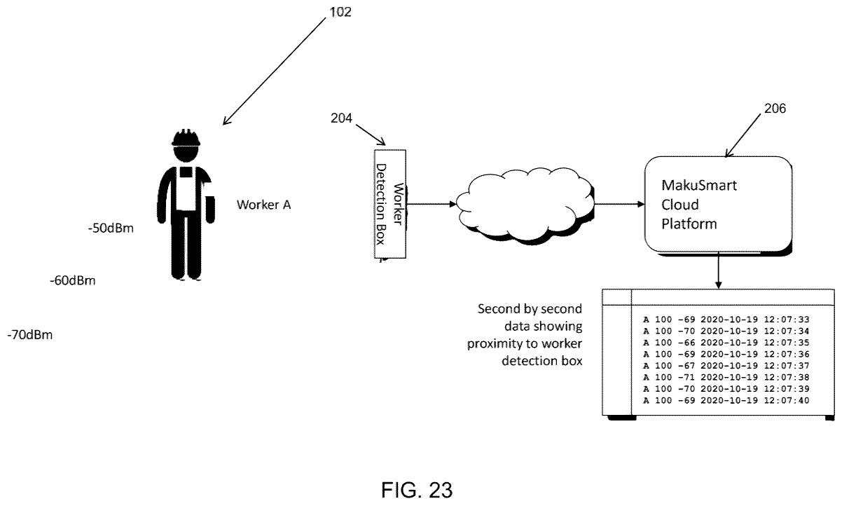

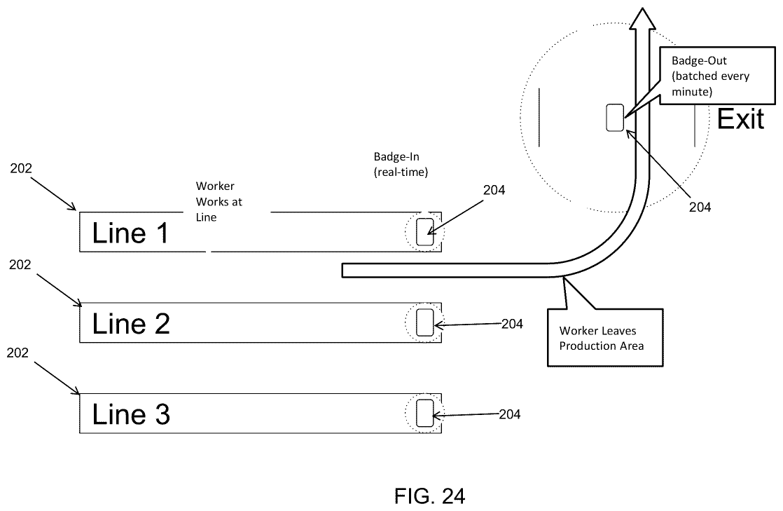

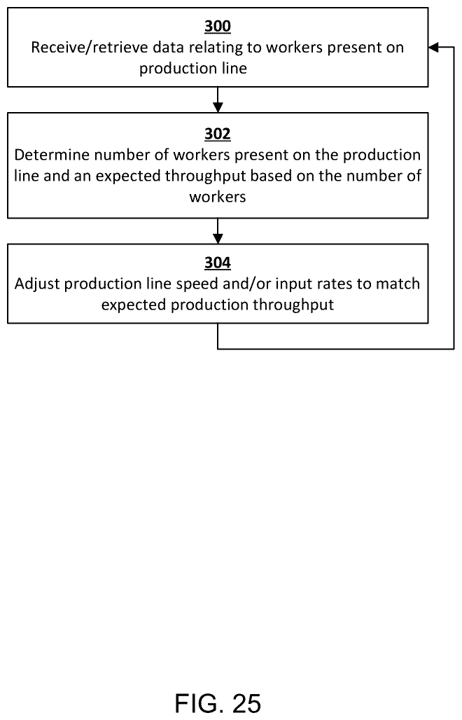

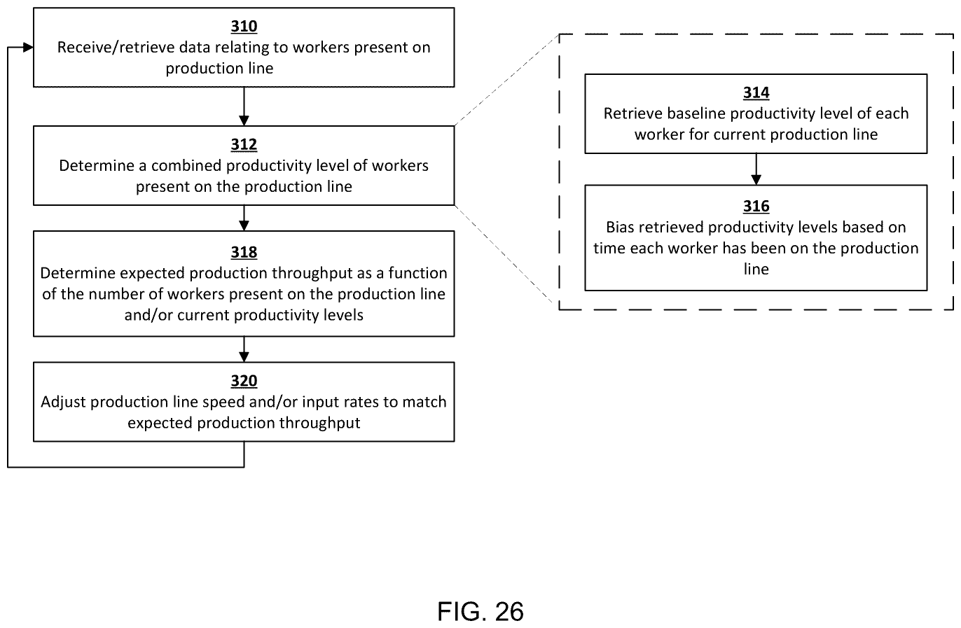

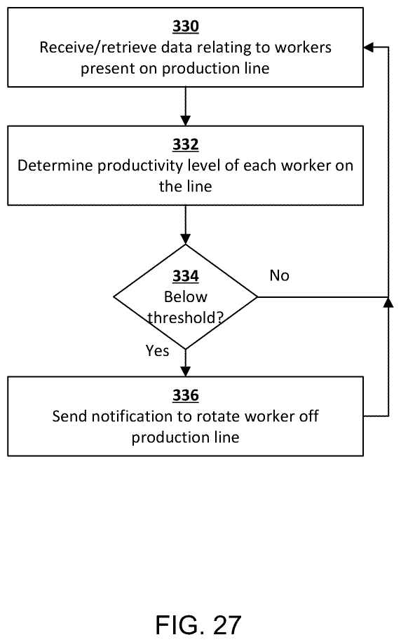

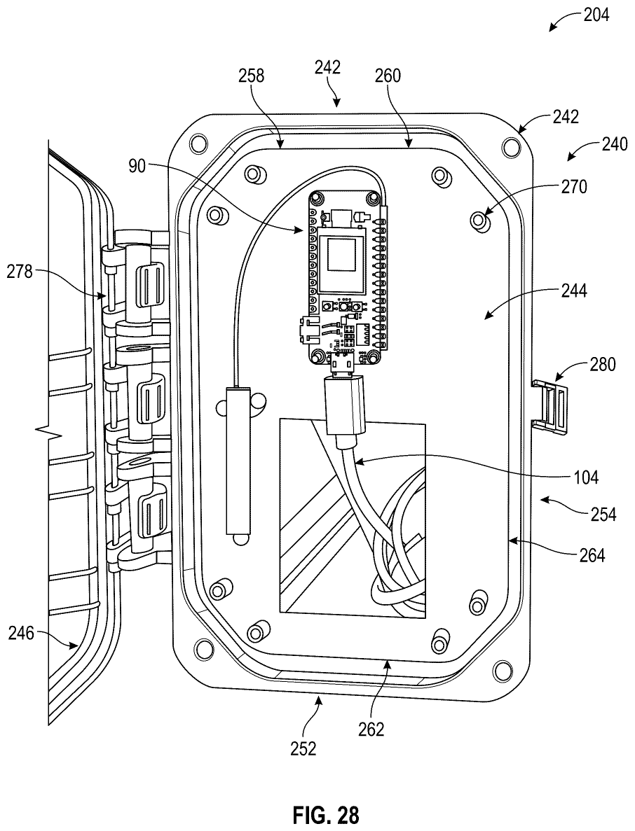

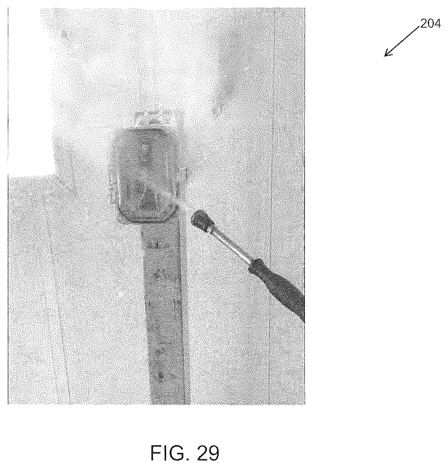

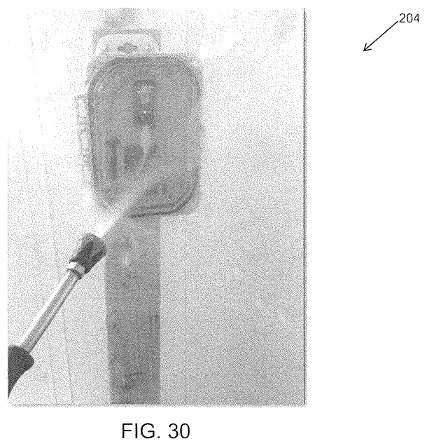

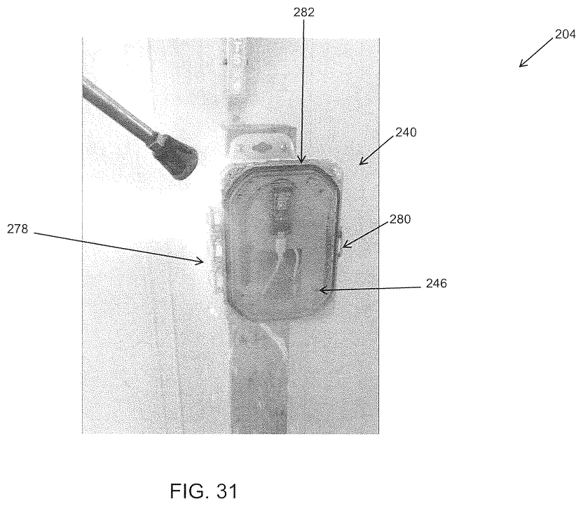

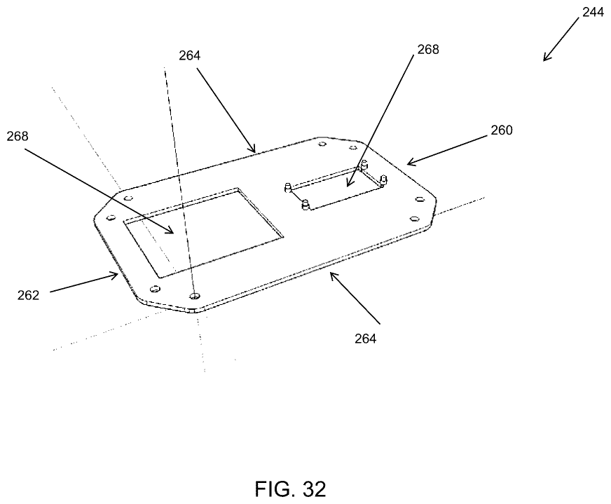

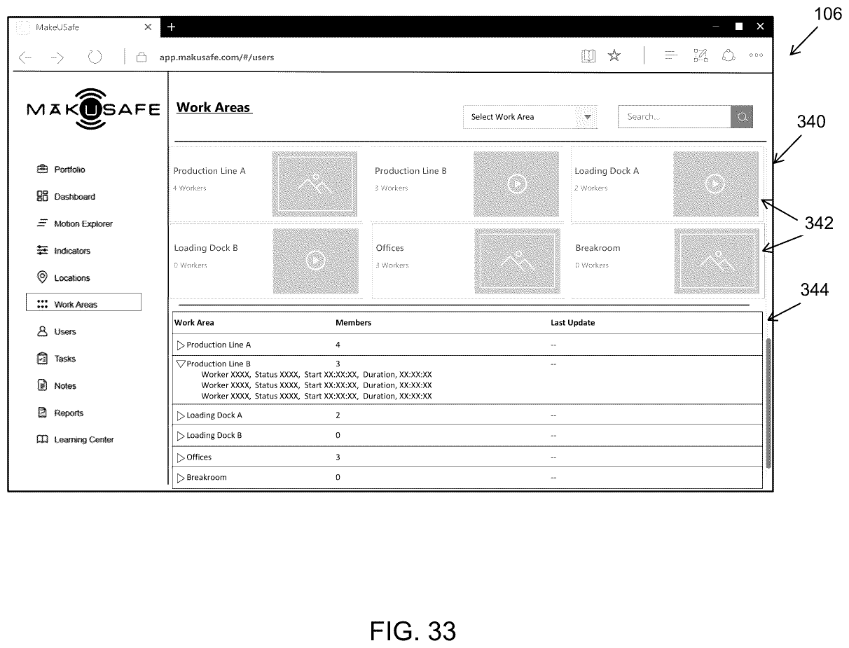

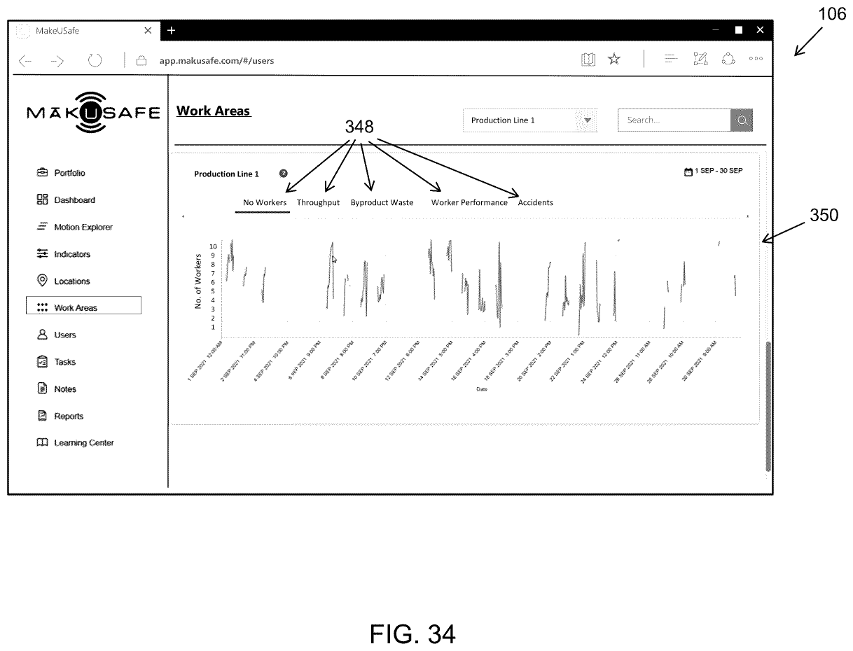

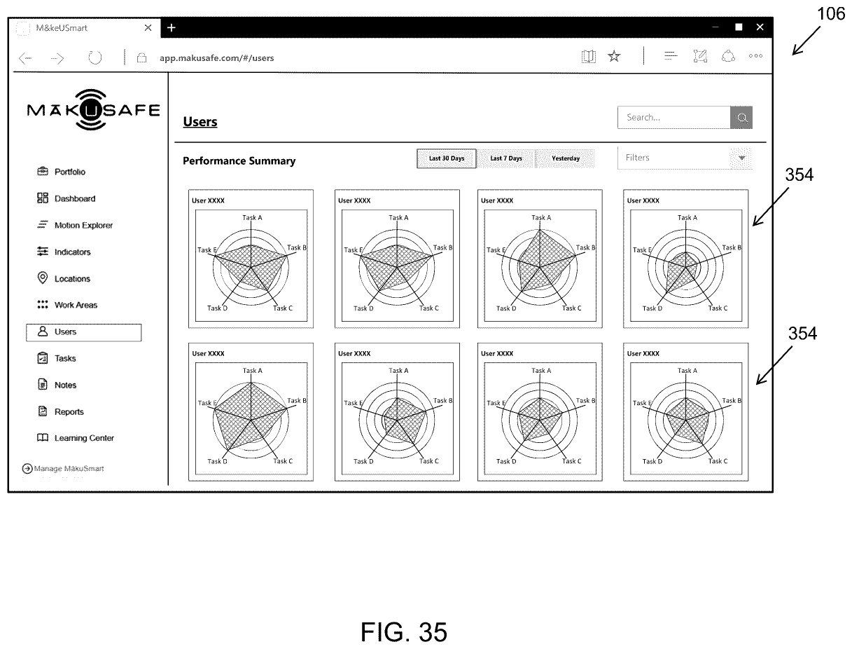

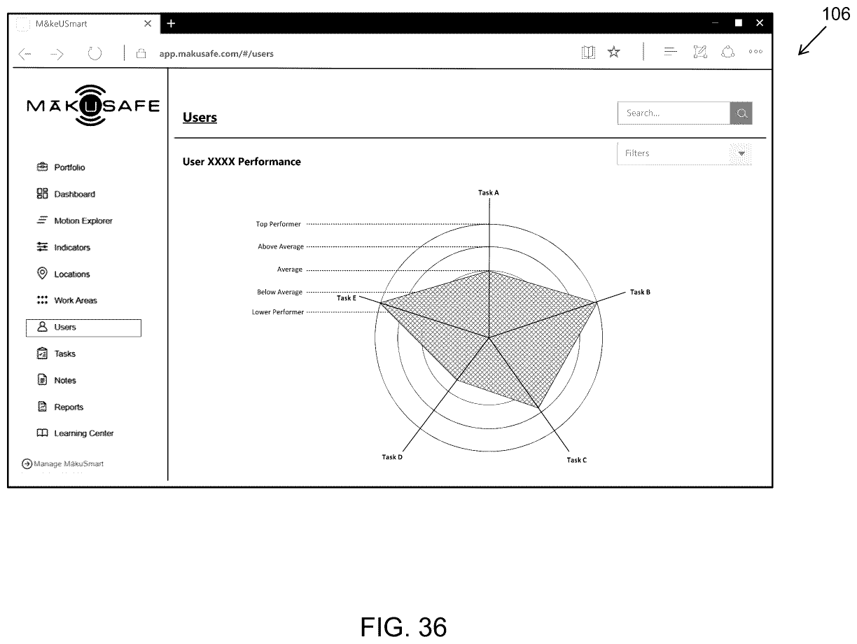

FIG. 9 is a block diagram illustrating components of a computing device that may be used to implement the methods and systems described in the document, as a client or plurality of clients. The computing system 900 represents variety kinds of computing devices such as smartphone, pad, laptop, and other similar computing devices. The computing system 900 described herein may be represented as the client 230 and 260. The computing system 900 includes RF (Radio Frequency) transceiver 910, baseband processor 920, Wi-Fi 930, Bluetooth/NFC (Near-Field Communication) 932, GPS 934, USB (Universal Serial Bus) 940, CPU 950, memories 960, audio 970, display/touchscreen 980 and camera modules 990. However, the specification of the client 260 is simpler than the one of the client 230. A lot of components in the client 260 may be removed, it depends on user's demand.

In one implementation, the CPU 950 may include multiple CPU cores, memory controller and peripheral bridge, which are interconnected together by using dedicated high-speed interfaces. The memory controller may be intended to connect with memories 960 by using variety buses. The peripheral bridge may be intended to connect with baseband processor 920, Wi-Fi 930, Bluetooth/NFC 932, GPS 934, USB 940, audio 970, display/touchscreen 980 and camera modules 990 by using variety buses.

The RF transceiver 910 may be a component to transmit and receive RF signals through antennas and extract digital signals coupled to the baseband processor 920, which may include a signal processing unit of baseband supporting variety protocols such as 3G, 4G and 5G (5th generation mobile networks). The baseband processor 920 may also be intended to process data sent from the CPU 950 and then transfer them to the RF transceiver 910 in order to transmit data to the network 220 through the antennas. The Wi-Fi 930 may be a component supporting a wireless networking technology to connect with the network 220. The Bluetooth/NFC 932 may be a component using wireless technologies to exchange data between the CPU 950 and desktop or mobile devices such as headsets, mouse, keyboard, printer, card reader and other similar devices.

The GPS 934 may be a component to receive location-based information and additional data from satellites through an antenna and transmit them to the CPU 950. The USB (Universal Serial Bus) 940 may be a component to establish a connection between the CPU 950 and external USB devices such as mouse, keyboard, printer, power charger and other similar devices. The memories 960 may be implemented as variety computer-readable media such as volatile memory, non-volatile memory and expansion memory. The CPU 950 may directly connect with the volatile memory and non-volatile memory by using dedicated buses. The non-volatile memory such as NAND andNOR Flash is a non-transitory computer-readable media, and may be used for storing a set of instructions, variety computer programs and apps. The system is intended to execute specific operations, computer programs and/or apps may be loaded from the non-volatile memory to the volatile memory such as SDRAM and other types of DRAM. The expansion memory is a non-transitory computer-readable media such as SSD for storing photos, videos and other information, and connect to the peripheral bridge of the CPU 950.

The audio 970 may be implemented by an audio codec and other circuits. The audio codec may be used for encoding audio digital data streams from the CPU 950 and decoding audio analog signals from a devices (e.g., microphone) converting audible sound to electrical signals. In one implementation, the audio 970 may convert the encoded audio digital data to analog audio signals and transmit them to an audible device (e.g., speakers or headsets). Moreover, the audio electrical signals may be converted to the encoded audio digital data for transmitting to the peripheral bridge of the CPU 950. The display/touchscreen 980 may be a component to drive a device of display and touchscreen. The computing system 900 may support a diversity of display technologies such as TFT (Thin-Film-Transistor Liquid Crystal Display) display, OLED (Organic Light Emitting Diode) display and other appropriate display technologies. Additionally, the computing system 900 may support variety touchscreen technologies such as resistive, surface acoustic wave, capacitive and other appropriate technologies.

The camera modules 990 may be implemented by one or more camera modules. In one implementation, the computing system 900 may support at least one front-facing and rear-facing camera modules. Additionally, the resolution of the rear-facing camera may be higher than the one of the front-facing camera. The camera modules 990 may be performed variety functions by executing a diversity of programs which are predetermined in the computing system 900, for example a face-recognition function. In another implementation, the at least one rear-facing camera may implement different functions by using specific lens such as telephoto lens, ultra-wide lens and other appropriate lens.

The foregoing implementations have been illustrated above, and may be implemented individually or in any combination. Additionally, variety modifications can be made to the foregoing implementations.

What is claimed is:

1. A method comprising:
Initiating, by an event of a healthcare plan, a plurality of clients and sensors in an area where a user arrives at;
detecting, by the plurality of clients and sensors, the user starts to engage in the event of the healthcare plan;
illustrating user's health status and the scenario of the event in the early stage of the event;
requesting, by the plurality of clients and sensors, a predetermined information of executing the event in the early stage of the event;
estimating the user's health status and the predetermined information in the early stage of the event;
providing a suggestion based on the estimation of the user's health status and the predetermined information in the early stage of the event;
confirming a decision with the user responsive to the acknowledgement of the suggestion from the user in the early stage of the event;
demonstrating, by the plurality of clients and sensors, entire process of the event responsive to the confirmation of the user in the early stage of the event;
configuring the plurality of clients and sensors involving in the event to periodically collect a diversity of data from the user's behavior of engaging in the event;
analyzing the diversity of data collected from the user's behavior of engaging in the event;
calculating the diversity of data and variety of values representing the user's health status; and
updating the user's health status after the calculations for the diversity of data and variety of values.

2. The method of claim 1, further comprising:
defining a target for each event in the user's healthcare plan based on the information of the user's health status and database of technologies;
creating processes and scenarios of events based on the information of the user's health status and database of technologies; and
adjusting dynamically an order of processes or scenario of events while the user is engaging in the events.

3. The method of claim 1, further comprising:
receiving periodically current location information provided by location-aware components of user's client, while the user's client is executing the event of the healthcare plan;
forming a list of venues suitable to carry out the event by determining the user arrives in a predetermined area enclosing the venues; and
illustrating a suggestion about an order of executing events in the venues of the list in the predetermined area to the user.

4. The method of claim 1, further comprising:
detecting the plurality of clients and sensors unable to collect information from the user's behavior while the user is engaging in the event;
interrupting the user's engagement for illustrating which part of the event the plurality of clients and sensors are unable to collect the information from; and
requesting the user to reply the information in order to continue the user's engagement in the event.

5. The method of claim 1, further comprising:
detecting the user's health status is deviating from the purpose of the event based on the series of information while the user is engaging in the event;
notifying the user about the deviation from the purpose of the event in order to interrupt the user's engagement;
performing a demonstration or termination of procedures of the event based on the analysis of the deviation; and
modifying the healthcare plan if the deviation is caused by improper processes of the event.

6. The method of claim 1, further comprising:
Initiating a plurality of instruments, clients and sensors in the area where the user locates;
measuring, by the plurality of instruments, clients and sensors, the user's health status after the event is completed; and
updating the user's health status and healthcare plan in response to the measurement for the user's health status.

7. A non-transitory computer-readable medium storing instructions that, when executed by a processor, cause the processor to implement the method comprising:
Initiating, by an event of a healthcare plan, a plurality of clients and sensors in an area where a user arrives at;
detecting, by the plurality of clients and sensors, the user starts to engage in the event of the healthcare plan;
illustrating user's health status and the scenario of the event in the early stage of the event;
requesting, by the plurality of clients and sensors, a predetermined information of executing the event in the early stage of the event;
estimating the user's health status and the predetermined information in the early stage of the event;
providing a suggestion based on the estimation of the user's health status and the predetermined information in the early stage of the event;
confirming a decision with the user responsive to the acknowledgement of the suggestion from the user in the early stage of the event;
demonstrating, by the plurality of clients and sensors, entire process of the event responsive to the confirmation of the user in the early stage of the event;
configuring the plurality of clients and sensors involving in the event to periodically collect a diversity of data from the user's behavior of engaging in the event;
analyzing the diversity of data collected from the user's behavior of engaging in the event;
calculating the diversity of data and variety of values representing the user's health status; and
updating the user's health status after the calculations for the diversity of data and variety of values.

8. The medium of claim 7, further comprising:
defining a target for each event in the user's healthcare plan based on the information of the user's health status and database of technologies;
creating processes and scenarios of events based on the information of the user's health status and database of technologies; and
adjusting dynamically an order of processes or scenario of events while the user is engaging in the events.

9. The medium of claim 7, further comprising:
receiving periodically current location information provided by location-aware components of user's client, while the user's client is executing the event of the healthcare plan;
forming a list of venues suitable to carry out the event by determining the user arrives in a predetermined area enclosing the venues; and
illustrating a suggestion about an order of executing events in the venues of the list in the predetermined area to the user.

10. The medium of claim 7, further comprising:
detecting the plurality of clients and sensors unable to collect information from the user's behavior while the user is engaging in the event;
interrupting the user's engagement for illustrating which part of the event the plurality of clients and sensors are unable to collect the information from; and
requesting the user to reply the information in order to continue the user's engagement in the event.

11. The medium of claim 7, further comprising:
detecting the user's health status is deviating from the purpose of the event based on the series of information while the user is engaging in the event;
notifying the user about the deviation from the purpose of the event in order to interrupt the user's engagement;
performing a demonstration or termination of procedures of the event based on the analysis of the deviation; and
modifying the healthcare plan if the deviation is caused by improper processes of the event.

12. The medium of claim 7, further comprising:
Initiating a plurality of instruments, clients and sensors in the area where the user locates;
measuring, by the plurality of instruments, clients and sensors, the user's health status after the event is completed; and
updating the user's health status and healthcare plan in response to the measurement for the user's health status.

13. A system comprising:
one or more processors; and
one or more non-transitory computer-readable media storing instructions that, when executed by the one or more processors, cause the system to perform operations comprising:
Initiating, by an event of a healthcare plan, a plurality of clients and sensors in an area where a user arrives at;
detecting, by the plurality of clients and sensors, the user starts to engage in the event of the healthcare plan;
illustrating user's health status and the scenario of the event in the early stage of the event;
requesting, by the plurality of clients and sensors, a predetermined information of executing the event in the early stage of the event;
estimating the user's health status and the predetermined information in the early stage of the event;
providing a suggestion based on the estimation of the user's health status and the predetermined information in the early stage of the event;
confirming a decision with the user responsive to the acknowledgement of the suggestion from the user in the early stage of the event;
demonstrating, by the plurality of clients and sensors, entire process of the event responsive to the confirmation of the user in the early stage of the event;
configuring the plurality of clients and sensors involving in the event to periodically collect a diversity of data from the user's behavior of engaging in the event;
analyzing the diversity of data collected from the user's behavior of engaging in the event;
calculating the diversity of data and variety of values representing the user's health status; and
updating the user's health status after the calculations for the diversity of data and variety of values.

14. The system of claim 13, further comprising:
defining a target for each event in the user's healthcare plan based on the information of the user's health status and database of technologies;
creating processes and scenarios of events based on the information of the user's health status and database of technologies; and
adjusting dynamically an order of processes or scenario of events while the user is engaging in the events.

15. The system of claim 13, further comprising:
receiving periodically current location information provided by location-aware components of user's client, while the user's client is executing the event of the healthcare plan;
forming a list of venues suitable to carry out the event by determining the user arrives in a predetermined area enclosing the venues; and
illustrating a suggestion about an order of executing events in the venues of the list in the predetermined area to the user.

16. The system of claim 13, further comprising:
detecting the plurality of clients and sensors unable to collect information from the user's behavior while the user is engaging in the event;
interrupting the user's engagement for illustrating which part of the event the plurality of clients and sensors are unable to collect the information from; and
requesting the user to reply the information in order to continue the user's engagement in the event.

17. The system of claim 13, further comprising:
detecting the user's health status is deviating from the purpose of the event based on the series of information while the user is engaging in the event;
notifying the user about the deviation from the purpose of the event in order to interrupt the user's engagement;
performing a demonstration or termination of procedures of the event based on the analysis of the deviation; and
modifying the healthcare plan if the deviation is caused by improper processes of the event.

18. The system of claim 13, further comprising:
Initiating a plurality of instruments, clients and sensors in the area where the user locates;
measuring, by the plurality of instruments, clients and sensors, the user's health status after the event is completed; and
updating the user's health status and healthcare plan in response to the measurement for the user's health status.
